(19) United States
(12) Patent Application Publication
(10) Pub. No.: US 2007/0192888 A1
(43) Pub. Date: Aug. 16, 2007

(54) Methods for breeding plants

(76) Inventors: Graeme David Richards, New South Wales (AU); George Orel, New South Wales (AU)

Correspondence Address: DYKEMA GOSSETT PLLC, 10 S. WACKER DR., STE. 2300, CHICAGO, IL, 60606, US

(21) Appl. No.: 10/573,341

(22) PCT Filed: Sep. 27, 2004
(86) PCT No.: PCT/AU04/01327

§ 371 Date: Feb. 27, 2007

(30) Foreign Application Priority Data
Sep. 26, 2003 (AU) 2003905277

(51) Int. Cl. A01H 1/02; C12Q 1/68

(52) U.S. Cl. 800/260; 800/284; 435/6

(57) ABSTRACT

The present invention provides a method for generating a woody perennial breeding line, comprising: a) selecting one or more woody perennial plants comprising at least one allele associated with male sterility; b) selecting one or more woody perennial plants which are capable of hybridisation with the plant(s) selected in step (a) and which comprise at least one allele associated with at least one target inheritable trait; C) crossing the one or more plants selected in step (a) with the one or more plants selected in step (b); d) selecting progeny plants which have one or more desired traits and which comprise at least one allele associated with male sterility and at least one allele associated with the at least one target trait; e) selecting progeny plants which are homozygous for male sterility, which comprise at least one allele associated with the at least one target trait and which have one or more desired traits. Male sterile woody perennial breeding lines generated by the methods of the invention are also provided.

### TECHNICAL FIELD

This invention relates to methods for breeding woody perennial plants, such as horticulturally important trees, and plants produced by these methods.

### BACKGROUND OF THE INVENTION

Environmental adaptation is of key importance in the breeding of woody plants. As our environment changes, both geographically and economically, we need to have the ability to readapt our crop plants to better suit the new environment(s). In many instances, this readaptation will require incorporation of one or more characteristics not currently present in an otherwise desirable variety of plant, and therefore cross-breeding with another plant which possesses the desired characteristic(s) may be necessary.

In general, plant breeding for woody perennials involves cross-pollination, and this typically results in offspring which are heterozygous for many characters, and/or of unknown genetic make up, particularly in the event of open pollination where the pollen parent may be unidentifiable.

Heterozygosity greatly diminishes the chances for producing a desired recombination of characters in a single plant. For example, when attempting to incorporate one or more target characteristics from a plant variety into an existing variety with an otherwise desirable set of inheritable traits, the plant variety providing the target characteristic(s) will often not possess as desirable a set of inheritable traits as the other plant, and offspring resulting from a cross between two such plants will typically possess intermediate, and often unacceptable traits.

Re-acquisition of desirable traits will then require an extensive program of back-crossing the progeny with one or more desired parentage lines, testing at each stage for incorporation of the target characteristic(s). This requires large progenies for selection because inadequate numbers almost certainly guarantee failure in any breeding program. The need for adequate growing space for seedlings and the typically long reproductive/juvenile cycle of woody perennials are costly in land and time.

Species capable of self-fertilising, such as peach and nectarines, also exist, and this also may complicate a breeding program.

Care must therefore be taken in a breeding program to ensure that only desired crosses occur.

In current technology, one method used for controlling the nature of crosses between varieties is to hybridise plants manually in combination with “artificial emasculation” of the anthers from the flowers of cultivated plants. Such techniques are very time consuming, and prone to error (e.g. missing emasculation, self crossing within one plant, etc).

There is therefore a need in the art for new methods for breeding woody perennial plants.

### SUMMARY OF THE INVENTION

The present invention provides novel methodology which permits accurate hybridisation, and which can also be conducted without manual intervention, in greenhouse conditions, or in isolation. The methods of the invention allow for a more directed and accelerated approach towards incorporation of target characteristics into woody perennial plants.

In the methods of the invention, male sterility is incorporated into selected plant breeding lines so as to facilitate the directed incorporation or alteration of characteristics in a woody perennial plant.

According to one embodiment of the invention, there is provided a method for generating a woody perennial breeding line, comprising:

a) selecting one or more woody perennial plants comprising at least one allele associated with male sterility;

b) selecting one or more woody perennial plants which are capable of hybridisation with the plant selected in step (a) and which comprise at least one allele associated with at least one target trait;

c) crossing the one or more plants selected in step (a) with the one or more plants selected in step (b);

d) selecting progeny plants which have one or more desired traits and which comprise at least one allele associated with male sterility and at least one allele associated with the at least one target trait;

e) selecting progeny plants which are homozygous for male sterility, which comprise at least one allele associated with the at least one target trait and which have one or more desired traits.

The plants resulting from this method may then form the basis for a breeding line which can be used to insert or alter one or more target characteristics/traits in a desired plant variety while maintaining other desirable traits in that variety, in an efficient manner in terms of both time and labour. In particular, by incorporating male sterility into woody perennial germplasm of known genotype with respect to a desired set of inheritable traits, the entire manual process of “artificial emasculation” is by-passed.

Male sterile woody perennial breeding lines produced by the methods of the invention are also provided.

According to another embodiment of the invention, there is provided a method for generating a woody perennial variety comprising one or more target inheritable traits, comprising crossing a first selected woody perennial plant variety with a desired set of inheritable traits, but lacking one or more target traits, with a second selected woody perennial plant which is homozygous for male sterility, and homozygous for at least one allele associated with the one or more target traits. Woody perennial varieties generated by this method are also provided.

### BRIEF DESCRIPTION OF THE DRAWINGS

FIG. 1 provides a cladogram in respect of relatedness between the following members of the genus Prunus (mostly cherries or related): P. avi=Prunus avium; P. camp=Prunus campanulata; P. ser=Prunus serotina; P. turn=Prunus turnerana; P. weird=unidentified very low chill requirement Prunus sp. from China (possibly Prunus ceracoides); P. yun=unidentified very low chill requirement Prunus sp. from the Yunan province in China (possibly Prunus ceracoides); P. viet=unidentified very low chill requirement Prunus sp. From Vietnam (possibly Prunus ceracoides); P. 1 inch=unidentified very low chill requirement Prunus sp. from the Yunan province in China (possibly Prunus ceracoides); P. UWS=low chill flowering hybrid (P. avium×P. campanulata); and the trees denoted by T are unidentified low chill requirement Prunus spp. (cherries) of unknown provenance.

FIG. 2 provides a dendrogram in respect of relatedness between the members of the genus Prunus identified in FIG. 1.

FIG. 3 provides a table of genetic distances for the cherry species identified in FIGS. 1 and 2.

FIG. 4 provides a schematic/flow diagram for the methods of the invention for generating male sterile breeding lines incorporating one or more target traits (sub-acid flesh exemplified), along with desirable inheritable traits. Subacid/honey flesh phenotype is a single dominant gene. At any stage pollen polymixes, carefully selected for the desired traits, can be used. At each step good selections can be obtained depending on the recurrent parent (backcross) and whether they are homozygous for desirable traits.

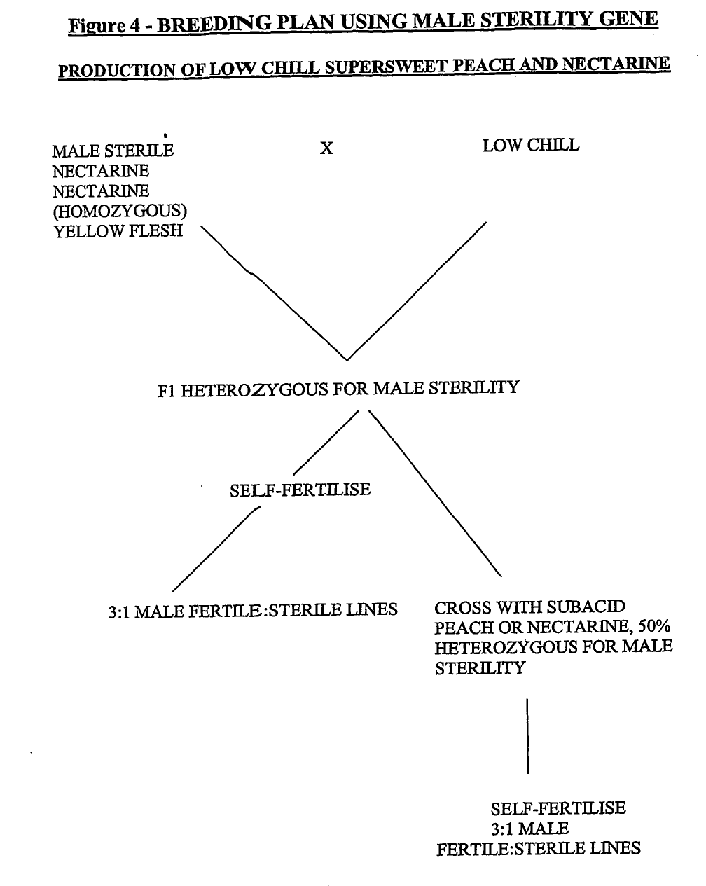

### DETAILED DESCRIPTION OF THE INVENTION

Definitions

“Allele”—Any one of a series of two or more different genes that occupy the same position (locus) on a chromosome. Since autosomal chromosomes are paired, each autosomal locus is represented twice. If both chromosomes have the same allele, occupying the same locus, the condition is referred to as homozygous for this allele. If the alleles at the two loci are different, the individual or cell is referred to as heterozygous for both alleles.

“Allele associated with” as used herein in conjunction with male sterility or target trait(s), means a gene which by itself, or in combination with other genes, codes for male sterility or the target trait(s) respectively. Thus, a plant having at least one allele associated with a given characteristic, such as male sterility or a target trait, means that the plant may be heterozygous or homozygous at least one gene locus which, by itself, or in combination with other gene loci, governs expression of that characteristic. Where a combination of loci are involved in expression of a given characteristic, expression of the characteristic may be quantitative or absolute, depending on the nature of the interaction between the respective gene products and expression of the characteristic.

“Chilling requirement”, “low chill” and “high chill” as used herein, relates to the length of time for which a deciduous plant must be exposed to a certain maximum temperature before normal budbreak, flowering and growth. Exposure to chilling temperatures is necessary to overcome dormancy in deciduous plants, after which normal bud break, flowering and growth can proceed once growing conditions are favourable. The minimal necessary duration of chilling length for any particular variety is known as the chilling requirement for that variety. Temperatures effective in satisfying the chilling requirement normally range from 0° C. to 10° C., with the optimal temperature being approximately 5-7.2° C. The chilling requirement value cited represents that minimum number of chilling hours required to break dormancy in 50% of the flower/leaf buds.

For example, peach varieties vary greatly in the number of chilling hours required from less than 200 hours (“Low Chill”) to over 1,000 hours (“High Chill”). The lower the chilling requirement, the earlier the tree will begin growing once temperatures are warm enough.

“Comprising”—in the context of this specification, the term “comprising” means “including principally, but not necessarily solely”. Variations of the word “comprising”, such as “comprise” and “comprises”, have correspondingly varied meanings.

Existing Breeding Methods

In general, plant breeding involves cross-pollination. However, many woody perennials, such as stone fruit like peach and nectarines, may be self-fertilisers. Cross-pollination with undesired plants and self-pollination are undesirable in breeding programs, and therefore care must be taken in the breeding program to ensure the right crosses are made.

In addition, the resulting progeny resulting from hybridisation will be heterozygous for many characters. Heterozygosity greatly diminishes the chances for producing a desired recombination of characters in a single plant, and therefore acquiring a desired phenotype or genotype will typically require large progenies for selection because inadequate numbers almost certainly guarantee failure in any breeding program. Also, the reproductive cycle for many woody perennials, such as stone fruit, is long, typically 3 to 10 years or more from seed to fruiting. The need for adequate growing space for seedlings and the long reproductive cycle are costly in land and time.

To increase control over the breeding process, traditional breeding techniques aim to create homozygosity in key desirable characteristics (see Table 1, next page, for traits relevant to stone fruit).

To avoid random events, a process of “artificial emasculation” is commonly employed whereby the anthers of the flowers that have not yet opened, are removed. The technique of “artificial emasculation” has been well described in the literature. For example; this methodology is described in: the USDA Year Book, 1937; “Advances in Fruit Breeding” (Janick, J. and Moore, J. N., eds., Purdue University Press, 1975); and “Methods in Fruit Breeding” (Moore, J. N. and Janick, J., eds., Purdue University Press, 1983).

The process of artificial emasculation inherently includes a number of disadvantages, including inefficiency (time and labour intensive) and propensity to error (e.g. missing emasculation, self crossing within one plant, etc)

The use of a male sterility gene in plant breeding has been used in the production of maize seed, where using male sterile inbred lines as female parents in certain hybrids does away with the need for detasselling as the tassels on male sterile plants shed no pollen. Seed production of hybrids based on male sterile inbred lines is thus an advantageous proposition for the commercial seed grower.

TABLE 1
Common desirable characteristics in stone fruit
(see also “Advances in Fruit Breeding” (Janick, J. and
Moore, J. N., eds., Purdue University Press, 1975))

| | |
|---|---|
| Character | Symbol |

Tree

| | |
|---|---|
| With anthocyanins/Anthocyanless | An/an |
| Tall, normal/Brachytic dwarf | Dw/dw |
| Tall, normal/Bushy | Bu₁/bu₁ |
| | Bu₂/bu₂ |
| Normal/Albino | C/c |
| Resistant/Susceptible to M. incognita | (Mi/mi)^{y} |
| Resistant/Susceptible to M. javanica | (Mj₁/mj₁)^{y} |
| | (Mj₂/mj₂) |

Foliage

| | |
|---|---|
| Red leaf/Green leaf | Gr/gr |
| Smooth/Wavy leaf margin | Wa/wa |
| Glandular/Eglandular foliage | E/e |

Flower

| | |
|---|---|
| Nonshowy/Showy | Sh/sh |
| Large/Small showy flowers | L/l |
| Coloured/White | W/w |
| Pink/Red | R/r |
| Dark pink/Light pink | P/p |
| Single/Double | D₁/d₁ |
| Fewer/More extra petals | Dm₁/dm₁ |
| | Dm₂/dm₂ |

Fruit

| | |
|---|---|
| White flesh/yellow flesh | Y/y |
| Pubescent skin/glabrous | G/g |
| Freestone/clingstone | F/f |
| Soft melting flesh/firm melting | St/st |
| Melting flesh/nonmelting flesh | M/m |
| Saucer shape/non-saucer | S/s |

By incorporating male sterility gene into woody perennial germplasm, the lengthy and inefficient manual process of “artificial emasculation” can be by-passed.

Any desired attributes may be incorporated into woody perennial varieties quickly and efficiently using this methodology, particularly where male sterility is incorporated into breeding lines homozygous for a range of other desirable characteristics.

Important elements of this technique include:

1. Male sterility gene in woody perennial plants with desired combination of attributes;

2. Use of natural vectors for pollination in controlled non-out crossing conditions;

3. No hand emasculation;

4. Managing flowering time (concurrent flowering of all plants);

5. Reduction of juvenile phase by chilling fruit kernels and pre-germination under refrigerated conditions;

6. Accurate evaluation due to forced uniformity of plant development;

7. Use of male sterility gene as a marker to track use of plants bred by this novel method.

In particular, development of woody perennial breeding lines which axe male sterile, homozygous for at least one allele associated with one or more target inheritable traits, and which are of known genotype for a set of desired, typically economically important, traits should overcome one or more of the above identified problems and provide a more efficient, directed means for improving or changing existing woody perennial varieties as required by new or changing markets/environments.

Methods for Generating Trait-Targeted Male Sterile Breeding Lines

According to the first embodiment, the present invention provides a method for generating a woody perennial breeding line, comprising:

a) selecting one or more woody perennial plants, each of which comprises at least one allele associated with male sterility;

b) selecting one or more woody perennial plants which are capable of hybridisation with the plant selected in step (a) and which comprise at least one allele associated with at least one target trait;

c) crossing the one or more plants selected in step (a) with the one or more plants selected in step (b);

d) selecting progeny plants which have one or more desired traits and which comprise at least one allele associated with male sterility and at least one allele associated with the at least one target trait;

e) selecting progeny plants which are homozygous for male sterility, which comprise at least one allele associated with the at least one target trait and which have one or more desired traits.

Selection of plants in steps (a), (b), (d) and (e) may be carried out by mere observation of the phenotype of the plants themselves, or their progeny (either through self-fertilisation or hybridisation with another plant of known characteristics), by polynucleotide analysis for known genetic markers, or by a combination of these methods.

The ability of the plants of steps (a) and (b) to hybridise may be determined empirically through trial and error, cladistic analysis, or other suitable method. Cladistic analysis, typically by analysis of banding patterns of extracted DNA, is most advantageous for selecting the plants in terms of economy of resources and time. DNA analysis may be carried out by any suitable method as known in the art. Typically the DNA analysis is carried out by RAPD (Rapid Amplified Polymorphic DNA) or Inter Simple Sequence Repeat (ISSR) DNA fingerprinting analysis. Advantageously, RAPD or ISSR analysis, and cladistic analysis may be carried out as per the improved methods described herein.

Under a number of circumstances, only one plant will be selected in step (a), step (b), or both. For example, this may be necessary where a plant selected in either of steps (a) or (b), or both possesses a particularly desirable set of inheritable traits, such as at least one allele associated with the at least one target trait and/or at least one allele associated with male sterility, and this combination is not readily available from other plants. Accordingly, the cross of step (c) will typically result in an F₁ progeny which is substantially homogenous genotypically, particularly if only one woody perennial plant is selected in both steps (a) and (b).

According to an aspect of the above method, step (d) may further comprise allowing at least one of the selected F₁ progeny plants, if heterozygous for male sterility, to self-fertilise to create an F₂ progeny for further selection. The F₂ progeny will typically possess a spread of genotypes inherited from the plant(s) of step (a), the plant(s) of step (O), or both, and the F₂ progeny plants with the most desirable genotypes, including at least one allele associated with male sterility and at least one allele associated with the one or more target traits will then be the subject of further breeding or selection.

According to another aspect, one or more of the F₁ progeny may be fertilized with a mixture of pollen obtained from a plurality of the selected F₁ progeny. The F₂ progeny will typically possess a spread of genotypes inherited from the plant(s) of step (a), the plant(s) of step (b), or both, and the F₂ progeny plants with the most desirable genotypes, including at least one allele associated with male sterility and at least one allele associated with the one or more target traits will then be the subject of further breeding or selection.

According to another aspect, step (d) may further comprise crossing at least one of the selected F₁ progeny plants with one or more woody perennial plants of known genotype with respect to a desired set of inheritable traits and which comprises at least one allele associated with male sterility to create an F₂ progeny for further breeding or selection.

The above aspects of the method of the invention will advantageously provide an F₂ progeny with greater genetic diversity to select from compared to the F₁ progeny where only one plant is selected in step (a), step (b), or both, whereby the cross of step (c) may result in an F₁ progeny which is substantially homogenous genotypically, particularly if only one woody perennial plant is selected in both steps (a) and (b).

According to an aspect of the above methods of the invention, the one or more woody perennial plants selected in step (a) are male sterile—that is, they are homozygous for male sterility. Such plants will also provide greater certainty as to the genotype of F₁ plants, and further generations, with respect to male sterility. Because the one or more woody perennial plants of step (a) are male sterile in this aspect of the invention, and cannot therefore self fertilise, the one or more woody perennial plants of step (a) may be grown in an isolated block, with the one or more selected woody perennial plants of step (b) without the need to emasculate the flowers of the plant(s) of step (a).

According to another aspect of the methods of the invention, a plurality of woody perennial plants are selected in step (b), and the flowers of the woody perennial plant(s) selected in step (a) are fertilised with a mixture of pollen collected from the woody perennial plants of step (b).

The converse may also apply, and therefore according to another aspect, a plurality of woody perennial plants are selected in step (a), and the flowers of the woody perennial plant(s) selected in step (b) are fertilised with a mixture of pollen collected from the woody perennial plants of step (a). This aspect, clearly, cannot apply where the one or more plants selected in step (a) are male sterile.

In the methods of the invention where a plurality of plants are selected in step (a) or step (b) for crossing with a plant selected in step (b) or step (a) respectively, the plurality of selected plants may comprise a range of varieties, species, or even genera, so as to allow for incorporation of a broad gene pool into the resulting selection of progeny. Furthermore, the use of pollen mixtures obtained from a number of woody perennial varieties, species, or genera has been found to increase the likelihood of successful hybridisation, particularly where the seed parent and the pollen parent are of different species or incompatibility groups, such as occur in cherries (Prunus avium, and related species). Use of pollen mixtures is also advantageous for overcoming self-incompatibility such as occurs in, for example, plums (for example Prunus salicina and P. domestica) apricots (P. armeniaca) and almonds (P. amygdalus).

According to another aspect of the methods of the invention, the one or more woody perennial plants selected in step (a), step (b), or both are of known genotype for a set of desired inheritable traits. Advantageously such plants express, and/or are homozygous for at least one allele associated with, one or more of the traits within the set of desired inheritable traits, and even more advantageously are homozygous for at least one allele associated with each trait within the set of desired inheritable traits.

According to another aspect of the methods of the invention, the one or more plants of step (b) are at least heterozygous for male sterility. This will ensure a proportion of the progeny of the cross in step (c) being homozygous for male sterility—a ratio of 1:3 male sterile: non-male sterile where the plant(s) selected in step (a) and step (b) are heterozygous for male sterility; and a ratio of 1:1 male sterile: non-male sterile where the plant(s) selected in step (a) are homozygous for male sterility and the plants selected in step (b) are heterozygous for male sterility or the plant(s) selected in step (a) are heterozygous for male sterility and the plants selected in step (b) are homozygous for male sterility.

According to another aspect of the methods of the invention, all of the woody perennial plants employed in the methods are of the same species. According to this aspect, there would be no uncertainty as to the ability of the plants to hybridise except where the species are subject to self-incompatibility (such as in plums and apricots) or group incompatibility (such as in cherries). In the case of self- or group incompatibility, appropriate varieties for hybridisation should be selected so as to avoid the incompatibility, or mixtures of pollens from a selection of varieties should be employed.

According to another aspect of the methods of the invention, the one or more woody perennial plants of step (a) are of a different species and/or genus to the one or more woody perennial plants of step (b). This will be required where the at least one target trait is not available in the species of one of the parent plants of step (a) or step (b), or is more readily available in a different species. For example, target traits such as extreme low chill requirement and/or disease/pest resistance may be more readily available in certain non-commercial or semi- or non-domesticated species.

Examples of species which may be used for crossing with P. persica, P. persica var. nucipersica, P. persica var nectarina, P. avium, P. cerasus, P. domestica, P. salicina, P. armeniaca, or P. amygdalus may include a wide variety of known Pruntes spp. However, currently identified species of interest include:

| | | |
|---|---|---|
| P. mira | P. mandschurica | P. ansu |
| P. davidiana | P. brigantiaca | P. ceracifera |
| P. mume | P. domestica | P. salicina |
| P. armeniaca | P. simonii | P. americana |
| P. sibirica | P. mexicana | P. hortulana |
| P. angustifolia | P. munsoniana | P. umbellata |
| P. communis | P. persica | P. persica var nectarina |
| P. pumila | P. besseyi | P. humilis |
| P. ceracoides | P. avium | P. pseudocerasus |
| P. campanulata | P. serotina | P. turnerana (pygeum) |

These species may have varying chromosome numbers compared to the plant to which they are to be crossed, and could need chromosome doubling.

For example, in P. avium 2N=16, whereas in P. pseudocerasus 2N=32.

Species which may cross with pears (Pyrus sp.) may include, for example, Pyrus pyrifolia or Pyrus communis.

Species which may cross with apples (Malus domestica) may include, for example, M. domestica, M. asiatica, or M. formosana.

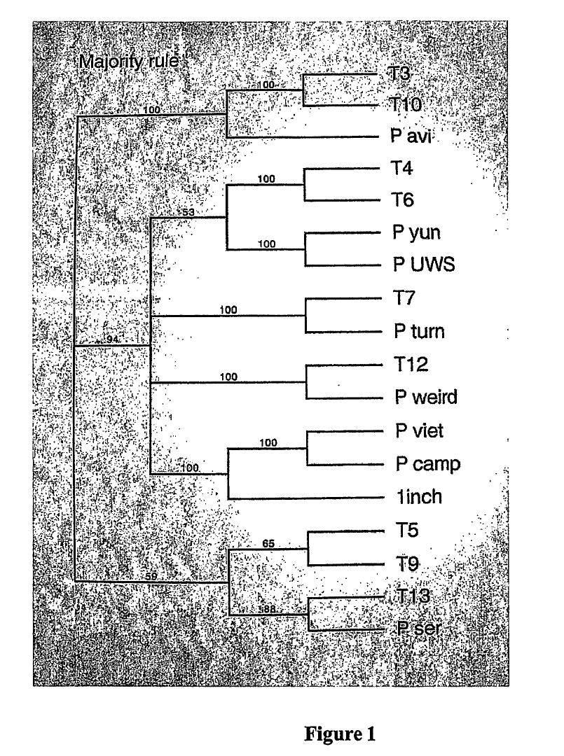

A cladogram, dendrogram and Table of Genetic Distances for a sample selection of cherries, which provide an indication of the likelihood of a successful hybridisation between these members of the Prunus genus, are provided in FIGS. 1 to 3.

Interspecific or intergeneric hybridisation may also be desirable where intermediate characteristics to those of either parent is desired, such as in plum×apricot, plum×peach, or apple×pear hybridisations. Interspecific or intergeneric hybridisation may also be used to transfer alleles associated with male sterility into a desired commercial species, optionally back-crossing the progeny to the desired commercial species so as to obtain what is effectively the desired commercial species with male sterility incorporated into its genome.

Mixtures of pollens from a selection of varieties for crossing with a different, non-domesticated or semi-domesticated species may improve the success of hybridisation.

In some instances where the plant(s) selected in step (a) are of a different species or genus to the plant(s) selected in step (b), there may be a difference in chromosome number which may result in completely or predominantly sterile progeny, if the plants succeed in hybridising to produce progeny at all. Thus, according to another aspect of the methods of the invention, the ploidy of the plant(s) of step (a) or step (b) is artificially increased to have a sufficiently similar chromosome number, preferably the same chromosome number as the other plant(s) to be used in the cross of step (c).

The chromosome number or ploidy of a woody perennial plant may be artificially increased by any suitable method as known in the art, typically including the use of colchicine or other spindle body formation inhibitor. Methods for increasing the ploidy in plants, including woody perennials, are described in a number of publications, for example, “Methods in Fruit Breeding” (Moore, J. N. and Janick, J., eds., Purdue University Press, 1983). Advantageously, the method used is the improved method for increasing ploidy levels in plants as described herein and/or as described in a co-pending International application titled “Method for Increasing Ploidy in a Plant” by Phytonova Pty Ltd, filed on 24 Sep. 2004, based on Australian provisional patent application No. 2003905278 by the University of Western Sydney, filed on 26 Sep. 2003, and incorporated herein in its entirety by cross-reference.

A combination of ploidy alteration and use of pollen polymixes, for interspecific or intergeneric crosses between plants of different chromosome number may also be used for further improvements in hybridisation efficiency.

Unfortunately, non-commercial or non-domesticated woody perennial species, such as low-chill and/or disease resistant germplasm contributors, may bring with them many undesirable traits. Even in the case of an intraspecific cross, or an interspecific cross between plants of commercial or domesticated varieties, a loss (partial or complete) of desired traits, such as fruit taste, flavour, size, colour or texture, or tree habit, may occur. Accordingly, backcrossing to one or more commercial varieties having a known genotype with respect to a desired set of commercial properties may be necessary so as to incorporate the desired commercial traits, along with male sterility and the at least one target trait into a breeding line.

Thus, according to an aspect of the methods of the invention, step (d) may further comprise one or more sequential back-crosses of one or more selected progeny plants with one or more woody perennial plants of known genotype with respect to a desired set of inheritable traits, and selecting resulting progeny plants which comprise at least one allele coding for male sterility and at least one allele associated with the at least one target trait and which are of known genotype for a desired set of inheritable traits. Selection of the one or more woody perennial plants for back-crossing to the selected progeny of step (d) may be carried out by mere observation of the phenotype of the plants themselves, or their progeny (either through self-fertilisation or hybridisation with another plant of known characteristics), and/or by polynucleotide analysis for known genetic markers. Also, the ability of these woody perennial plants to hybridise with the selected progeny of step (d) may be determined empirically through trial and error, cladistic analysis, or other suitable method. Again, cladistic analysis, typically through analysis of banding patterns of extracted DNA, by methods known in the art, or by the improved DNA and cladistic analysis methods described herein, is most advantageous in terms of economy of resources and time.

Typically, the one or more parental woody perennial plants used for backcrossing to the progeny of step (d), express a set of desired inheritable traits. More typically they are homozygous for at least one allele associated with one or more desired inheritable traits. Even more advantageously the one or more parental woody perennial plants used for backcrossing to the progeny of step (d) are homozygous for at least one allele associated with each trait within a set of desired inheritable traits.

Breeding lines developed by the methods of the invention which are homozygous for most economic traits helps hasten the development of new cultivars.

The one or more parental woody perennial plants used for backcrossing to the progeny of step (d), may be heterozygous for male sterility, or homozygous male sterile plants where the progeny plants are heterozygous for male sterility.

The parental woody perennial plants used for backcrossing to the progeny of step (d), may be of the same species, or be the same, or of the same variety as the one or more woody perennial plants of step (a), step (b), or both.

According to another aspect of the methods of the invention, the at least one target trait is selected from the group comprising: low chill requirement; high chill requirement; disease/pest resistance; fruit development period; fruit acidity; fruit shape; fruit size; fruit flesh texture; fruit total solids (sugars); fruit-skin pigmentation; fruit flesh pigmentation; fruit skin pubescence; stone adhesion to the fruit; tree habit; tree size; tree growth rate; spur morphology/habit; pedicel length; pedicel thickness; suture presence/absence.

The target trait may be polygenic—that is, one which is governed by a number of separate genetic loci. Examples include chilling requirement, disease/pest resistance, plant and/or fruit size, amongst others. Advantageously, the allele(s) for this trait, as provided by the plant(s) selected in step (b) is/are complementary to any allele(s) associated with the trait which may be provided by the plant(s) selected in step (a).

According to another aspect of the methods of the invention, the at least one target trait comprises low chill requirement. Insertion of low chill requirement into stone fruit such as peaches, nectarines, plums, apricots and cherries will result in plants which can be grown in warmer climates than currently known while not compromising their flowering and fruiting/yielding characteristics, potentially opening up new or more effective fresh fruit markets. Crossing/hybridising plants with different chilling requirements will require storage of pollen and artificial fertilisation of flowers.

It is possible to continuously select for lower and lower chill (which is a polygenic trait) as segregation allows for the shuffling of genes in combinations which allow for this. Unfortunately the increments in lowering chill requirement are generally small and a major shift, if necessary, is only currently possible by using either wild or semi-domesticated germplasm, or related species.

The present invention also involves the recognition of several forms of the species being dealt with or related species, for example Prunus species which have a very low-chill requirement which will artificially hybridise. Such low-chill species include: Prunus campanulata, P. ceracoides and P. angustifolia.

Low-chill requiring plants of non-domesticated germplasm typically flower early and generally mature their fruits quickly with the unfortunate consequence of a loss in at least fruit qualities of size and flavour.

Where the one or more low-chill woody perennial plants selected in step (b) are non- or semi-domesticated plants, backcrossing to one or more commercial varieties having a known genotype with respect to a desired set of commercial properties, as described above, may be necessary so as to achieve desired commercial traits including, for example, a long fruit development period gene, which allows the plant a longer time to produce carbohydrates for the fruit, in conjunction with male sterility, low chill requirement and possibly one or more additional target trait(s).

According to another aspect of the methods of the invention, the one or more woody perennial plants of step (b) express at least low chill and disease/pest resistance as target traits. Often, disease/pest resistance will also be more readily available from germplasm of a non-domesticated species or variety, and therefore backcrossing with a domesticated variety of known genotype with respect to a set of desired commercial traits may be required so as to achieve a male sterile breeding line incorporating the desired traits in combination with at least low chill requirement and disease/pest resistance.

Disease/pest resistance may be resistance to bacterial leaf spot, Shaka (Plum Pox Virus), bacterial canker, root rot, brown rot, peach canker, bacterial canker, bacterial blossom blight.

According to another aspect of the methods of the invention, the desired inheritable traits are selected from one or more of the group comprising: low chill requirement; high chill requirement; disease/pest resistance; fruit development period; fruit acidity; fruit shape; fruit size; fruit flesh texture; fruit total solids (sugars); fruit skin pigmentation; fruit flesh pigmentation; fruit skin pubescence; stone adhesion to the fruit; tree habit; tree size; tree growth rate; spur morphology/habit; pedicel length; pedicel thickness; suture presence/absence.

According to another aspect of the methods of the invention, all the plants are of the genus Prunus. More typically, the one or more woody perennial plants of step (a) are selected from peach (Prunus persica), nectarine (P. persica var nucipersica, P. persica var nectarina), plum (P. salicina and P. domestica), cherry (P. avium or P. cerasus), almond (P. amygdalus) or apricot (P. armeniaca) varieties.

According to another aspect the one or more woody perennial plants of step (a), step (b), or both are related to peaches, nectarines, plums, cherries, almonds or apricots, but are of a different species. For example, in the case of cherries, the plant(s) selected in step (a) may be selected from P. campanulata, P. ceracoides, P. pseudocerasus, in the case of peaches and nectarines, the plant(s) selected in step (a) may be selected from Prunus salicina, P. domestica, P. amygdalus, P. armeniaca, P. mira or P. davidiana (the latter two of which may provide Shaka resistance), and in the case of plums, the plant(s) selected in step (a) may be Prunus angustifolia, which provides low chill requirements.

Even more typically, the one or more woody perennial plants of step (a), step (b), or both are selected from peach or nectarine varieties.

Alternatively, the one or more woody perennial plants of step (a), step (b) or both are related to peaches or nectarines, but are of a different species. For example, Resistance to Shaka disease, as a target trait, may be found in P. mira and P. davidiana which are able to hybridise with peaches and nectarines.

According to another aspect of the methods of the invention in which the one or more woody perennial plants of step (a), the one or more woody perennial plants of step (b), or both are selected from peach, nectarine, plum, cherry, almond or apricot varieties, step (d) further comprises one or more sequential back-crosses of one or more selected progeny plants with one or more parental woody perennial plants which are selected from peach, nectarine, plum, cherry or apricot varieties, typically peach or nectarine varieties.

According to another aspect of the methods of the invention in which the one or more woody perennial plants of step (a), the one or more woody perennial plants of step (b), or both are selected from peach, nectarine, plum or apricot varieties, step (d) further comprises one or more sequential back-crosses of one or more selected progeny plants with one or more woody perennial plants which are related to peaches, nectarines, plums or apricots, but of a different species to the plant(s) selected in step (a), step (b) or both steps (a) and step (b).

According to another aspect of the methods of the invention, the one or more woody perennial plants of step (a) are selected from peach or nectarine varieties, and the one or more woody perennial plants of step (b) are sufficiently related to peaches or nectarines so as to be able to hybridise therewith, but are of a different species. According to this aspect, step (d) may further comprise one or more sequential back-crosses of one or more selected progeny plants with one or more parental woody perennial plants which are selected from peach or nectarine varieties, or sufficiently related species. Typically, the latter woody perennial plants and the one or more woody perennial plants of step (a) are of the same variety.

According to another aspect of the methods of the invention involving Prunus species, the one or more target traits comprise low chill requirement. Advantageously, the one or more woody perennial plants of step (b) also comprise at least disease/pest resistance, as a target trait. Typically, the one or more woody perennial plants of step (b) will be from, or derived from a non-domesticated or semi-domesticated species related to peaches, nectarines, plums, cherries, almonds or apricots, and the one or more woody perennial plants of step (a) will be from, or derived from peach, nectarine, plum, cherry, almond or apricot varieties.

According to another aspect of the invention, the one or more woody perennial plants of step (b) are selected from plum or apricot varieties. According to this aspect, step (d) may further comprise one or more sequential back-crosses of one or more selected progeny plants with the pollen of a plurality of woody perennial plants which are selected from plum or apricot varieties.

According to another aspect of the methods of the invention, the resulting woody perennial breeding line is male sterile, homozygous for a desired set of inheritable traits, and homozygous for at least one allele associated with at least one target trait.

Male Sterile Targeted Breeding Lines

According to another embodiment of the invention, male sterile woody perennial plant breeding lines homozygous for at least one allele associated with at least one target trait and being of known genotype with respect to a set of desired inheritable traits, generated by a method according to the invention are also provided.

According to an aspect of this embodiment, the male sterile woody perennial plant breeding line is a Prunus variety selected from peach, nectarine, plum, cherry, apricot or almond varieties.

According to another aspect of this embodiment, the male sterile woody perennial plant breeding line is the result of the cross of step (c) being an interspecific cross. The resulting plant may be a plumcot, a pleach, or other hybrid.

Alternatively, an interspecific hybrid resulting from an interspecific cross in step (c) may be back-crossed several times to a particular plant variety, which may be the same plant, or plant variety as selected in step (a) or step (b), so as to re-acquire the desired traits from the species originally selected in step (a) or step (b), thereby resulting in a plant which is technically a new species, but which is essentially the same species as the plant species selected in step (a) or step (b).

Thus, according to this aspect, where, for example the method involves Prunus species, the male sterile woody perennial plant breeding line may be essentially a Prunus variety selected from peach, nectarine, plum, cherry, apricot or almond varieties.

According to another aspect of this embodiment, the one or more target traits comprise at least low chill requirement, disease/pest resistance, or both.

According to another aspect of this embodiment, the resulting woody perennial breeding line is male sterile, homozygous for the desired set of inheritable traits, and homozygous for at least one allele associated with at least one target trait.

According to another embodiment of the invention, there is provided a method for generating a woody perennial variety comprising one or more target inheritable traits, comprising crossing a first selected woody perennial plant variety with a desired set of inheritable traits, or a group of plants sharing a set of desired inheritable traits, with a second selected woody perennial plant which is homozygous for male sterility, and homozygous for one or more target traits.

According to an aspect of this embodiment, the second plant is of known genotype with respect to the desired set of inheritable traits of the first selected woody perennial plant or group of plants sharing a set of desired inheritable traits.

The second plant may typically also express the desired set of inheritable traits of the first selected woody perennial plant or group of plants sharing a set of desired inheritable traits. Advantageously, the second plant may be homozygous for one or more of the desired set of inheritable traits of the first selected woody perennial plant, or group of plants sharing a set of desired inheritable traits, and even more advantageously be homozygous for each of the desired set of inheritable traits of the first selected woody perennial plant or group of plants sharing a set of desired inheritable traits.

According to this embodiment of the invention, the second woody perennial plant is typically a male sterile woody perennial plant breeding line developed by the methods described above.

According to an aspect of this embodiment, the first selected woody perennial plant variety with a desired set of inheritable traits, or group of plants sharing a set of desired inheritable traits, are planted in an orchard, surrounding the second plant.

According to another aspect of this embodiment, pollen from the first selected woody perennial plant variety with a desired set of inheritable traits, or a mixture of pollen from the group of plants sharing a set of desired inheritable traits, is used to artificially fertilise the flowers of the second plant.

According to another aspect of this embodiment, the plants used in the method are members of the genus Prunus. Typically, the plants are selected from Prunus plants which are, or are essentially peach, nectarine, plum, cherry, apricot or almond varieties Alternatively, at least one of the plants used for the method is a hybrid between two different Prunus species, for example, a plumcot or a pleach.

According to another aspect of this embodiment, the male sterility was inserted into the second woody perennial variety by means of recombinant DNA technology.

Woody perennial plant varieties generated by the above methods are also provided.

According to an aspect of this embodiment, the plant varieties generated by the above method are selected from Prunus plants which are, or are essentially peach, nectarine, plum, cherry, apricot or almond varieties. Alternatively, at least one of the plants used for the method is a hybrid between two different Prunus species, for example, a plumcot or a pleach.

According to another aspect, the resulting plant variety, or varieties, express one or more target traits selected from the group comprising: low chill requirement; high chill requirement; disease/pest resistance; fruit development period; fruit acidity; fruit shape; fruit size; fruit flesh texture; fruit total solids (sugars); fruit skin pigmentation; fruit flesh pigmentation; fruit skin pubescence; stone adhesion to the fruit; tree habit; tree size; tree growth rate; spur morphology/habit; pedicel length; pedicel thickness; suture presence/absence.

According to a another aspect, the resulting plant variety, or varieties, express at least low chill, disease/pest resistance, or both as target traits.

According to a further aspect, the resulting plant variety expresses heterosis for one or more target traits.

Selection of Plants for Hybridisation by Cladistic Analysis

The ability of two species to hybridise in the methods of the present invention may be determined by trial and error, but is more appropriately and efficiently determined by cladistic analysis. This typically requires analysis of the genetic material of the two plants, and may be carried out by any appropriate method as known in the art. Typically the DNA analysis is carried out by RAPD (Rapid Amplified Polymorphic DNA) analysis or Inter Simple Sequence Repeat (ISSR) DNA fingerprinting.

Advantageously, an improved method for DNA extraction and cladistic analysis as described below, which has been developed specifically for woody perennials is employed for determining the ability of two species to hybridise in the methods of the present invention.

DNA Extraction

The availability of fresh materials may determine the weight of individual samples.

Genomic DNA is extracted from fresh leaves (<1 g), using a method similar to that of Dellaporta et al (1983, as described by Wilkie et al 1997). Tissues are ground by hand in ceramic mortars with sand and liquid nitrogen and then mixed with 40 mL of rinse buffer (50 mM Tris, 100 mM NaCl, 100 mM EDTA, 1% PVP, pH7.5) and left for 30 mins at 4° C. After centrifugation for 10 min at 4 000×g, the supernatants are discarded, and the residue resuspended in 10 mL of digestion buffer (50 mM Tris, 100 nM NaCl, 100 mM EDTA, 0.5% SDS, pH7.5) and incubated at 65° C. for 15 min. Protein and carbohydrate are precipitated, together with dodecyl sulphate, by the addition of 900 μL of 10M potassium acetate, followed by incubation at 0° C. for 30 min. After centrifugation at 2 800×g for 15 min, the supernatant is removed to new tubes. Nucleic acid is precipitated by the addition of 2 volumes of 96% ethanol, removed from the liquid and rinsed twice with 70% ethanol.

A purification procedure using diatomaceous earth binding, adapted from the technique described by Gilmore et al. (1993) is then followed: 1.5 mL of a binding agent (50 mM Tris, 6M NaCl₄, 1 mM EDTA) was added and the mixture is incubated for 20 min at ambient temperature. 300 μL of a water suspension of acid-washed diatomite is added and followed by a further 30 min incubation. The mixtures are centrifuged at 550×g for 10 min and the supernatants discarded. The diatomite sediments (with bound DNA) are twice washed with 1.5 mL of 3:1 (binding agent: H₂O) and then once with 1.5 mL of 20 mM Tris, 2 mM EDTA and 2M NaCl in 25% ethanol, each wash being followed by centrifugation at 1 000×g for 10 min.

DNA is eluted with 300 μL of TE (10 mM Tris, 1 mM EDTA, pH 7.5) at 50° C. for 10 min. After centrifugation at 2 800×g and removal of the supernatants to new tubes, the DNA is precipitated by the addition of 2.5 volumes of 95% ethanol containing 120 mM sodium acetate, centrifuged at 2 800×g for 4 min and rinsed with 70% ethanol, then with 96% ethanol, before being dried under vacuum for 10 min. Purified DNA is redissolved in 500 μL of 0.1 TE.

The quality and quantity of extracted genomic DNA samples is assessed by electrophoresis in 1% agarose gels, after staining with ethidium bromide. Electrophoresis is used as a general diagnostic tool to ascertain the quality and quantity of genomic DNA extracted. It is also used to evaluate PCR products.

Horizontal Agarose Electrophoresis

Gel Electrophoresis is performed at 140V and 93 μA, for a period of 45 mins. Gels are prepared as follows:

2.5 g agarose is fully dissolved in 250 mL of electrophoresis buffer (45 mM Tris—borate, 1 Mm EDTA (Sambrook et al 1989)). The mixture is held on a high heat at 250° C. in a microwave oven for 3 mins, cooled to 55° C., then poured into a tray with gel combs in place. After 1 hour the hard gel is placed into the electrophoresis machine and immersed into an electrophoresis buffer.

Markers are prepared as follows:

0.5 μL of Promega pGem DNA Marker G174A, 9.5 μL of electrophoresis buffer (45 mM Tris—borate, 1 mM EDTA (Sambrook et al 1989)) and 2 μL of Promega Blue/Orange Loading Dye, 6×(G188A).

The DNA samples are prepared as follows:

5 μL of each individual DNA sample is mixed with 5 μL of electrophoresis buffer and 2 μL of 6×(G188A) dye. 12 μL of marker and 12 μL of each DNA sample are placed into the pertinent ‘wells’ within the gel. The previously calibrated Gel Electrophoresis Apparatus is run for 45 mins.

When the process is completed, a section of gel containing the DNA samples is cut out and placed into a vessel containing a solution of Ethidium bromide, 54 g Tris, 27.5 g Boric acid and 20 mL 0.5M EDTA, at pH 8.0. The vessel is placed onto an orbital shaker for a period of 1 hour to aid thorough and even staining. The stained piece of gel was retrieved and photographed.

Polymerase Chain Reaction (PCR)

A region of chloroplast DNA comprising the tRNA leucine (UAA) gene (trnL), the intron it contains, the tRNA phenylalanine (GAA) gene (trnF) and the intergenic spacer between trnL (5′ exon) and trnF, is amplified using the polymerase chain reaction (PCR) (Mullis and Faloona, 1987), using the primers A50272 and B49317 of Taberlet et al. (1991).

Amplification by PCR of the trnL/F region is performed in a HYBAID OMN-E thermocycler, using the following program: 5 min at 96° C.; and 30 sec at 96° C., 30 sec at 60° C. and 1 min at 74° C., repeated 30 times.

The reaction mixture contains 2.5 μL of 10×PCR buffer (PROMEGA #M1906), 1.5 μL 25 mM MgCl₂, 2 μL of ‘4dNTPs’ (2.5 mM each of dATP, dCTP, dGTP and dTTP), 6 μL of each of the two primers at a concentration of 20 μM, 37 μL H₂O and 0.2 μL Taq Polymerase (BIOTAQ from BIOLINE Co., 5 units/μL), for a total volume of 50 μL.

PCR products are purified using the CONCERT PCR Purification Kit (GibcoBRL Co.). DNA sequences can be ascertained using the ABI prism fluorescent dye-terminator system (Applied Biosystems, Foster City, Calif.).

Random Amplification of Polymorphic DNA (RAPD)

RAPD, a PCR based method is used to amplify short anonymous stretches of DNA which are then separated and visually reproduced by gel electrophoresis.

The protocol of Welsh and McClelland (1990) is followed. DNA fragments of interest are amplified using appropriately selected primers.

The reaction mixture for RAPDs consists of: 2 μL 10×PCR buffer, 2 μL MgCl₂ 25 mM, 2 μL 4 dNTP, 4 μL primer 20 mM, 10 μL H₂O and 0.2 μL of Taq polymerase (5 units μL, Promega). 2.5 μL of each respective DNA (quantity not estimated) was placed into each tube. The PCR was performed using a Corbett FTS 4 000 Thermal Sequencer and the following program: 96° C. for 3 min, then 40 repetitions of 96° C. for 1.5 min, 36° C. for 1.5 min, and 72° C. for 2.5 min, followed by 72° C. for 6 min.

PCR products are analysed by poly-acrylamide electrophoresis and revealed by silver-staining using a Gene Gel Exel 12.5/24 pre—cast gels, run on a GenePhor electrophoresis apparatus and stained with the PlusOne kit (all from Pharmacia, 100V, 2 hours). Images of the silver-stained gels are scanned directly into a computer and enlarged and printed for visual analysis.

Molecular sizes of identified bands are estimated by comparison with Promega ‘pGem DNA markers (#G174).

A binary number data matrix is constructed in which the absence of a band is denoted 0 and the presence of a band 1. The matrix is subjected to analysis using PAUP version 4.0b5 for Macintosh software package (Swofford, 2000) and MacClade (Maddison and Maddison, 1992).

Inter Simple Sequence Repeat (ISSR) DNA Fingerprinting

ISSR, another PCR based method may also be used to amplify short anonymous stretches of DNA which are then separated and visually reproduced by gel electrophoresis.

ISSR-PCR is carried out using a protocol based on that described by Briard et al. (2001). Each reaction mixture contains 2.5 μL of 10×PCR buffer (Promega #M190G), 2 μL of ‘4dNTPs’ (as above), 2.5 μL of 25 mM MgCl₂, 3.75 μL of the primer (AG)₈T₂ at a concentration of 20 μM, 15 μL H₂O, and 0.5 μL Taq Polymerase (Promega, 5 units/μL), and 2 μL of each respective DNA (quantity not estimated), for a total volume of 25 μL. The PCR was performed in a Corbett Research CP2-03 Thermal Sequencer, using the following program: 5 min at 94° C.; 35 cycles of 30 sec at 94° C.; 30 sec at 60° C. and 5 min at 72° C.

PCR products are analysed by electrophoresis through 2% agarose in TBE electrophoresis buffer (2.75 g/L boric acid, 5.4 g/L Tris and 10 mM EDTA), 5 V/cm for 2 hours, stained with ethidium bromide, and photographed under UV illumination.

A binary number data matrix is constructed in which the absence of a band is denoted 0 and the presence of a band 1. The matrix is subjected to analysis using PAUP version 4.0b5 for Macintosh software package (Swofford, 2000) and MacClade (Maddison and Maddison, 1992).

GeneGel Exel 12.5/24 Kit

The Polyacrylamide method yields better results compared to agarose-based methods, as the depiction of DNA bands is sharper, especially when dealing with ‘faint’ bands.

GeneGel Exel 12.5/24 Kit contains pre-cast gels (Stacking gel T=6%, C=3%, separating gel T=12.5%, C=2%) with pre-formed sample wells (6 μL).

The Kit also contains polyacrylamide strips (strip matrix polyacrylamide T=12%, C=3%) containing the buffer needed for electrophoresis. The anode buffer strip contains 0.45 mol/L Tris/Acetate, 4 g/L SDS and 0.05 g/L Orange G. The cathode buffer system contains 0.08 mol/Tris, 0.80 mol/L Tricine and 6 g/L SDS.

The kit forms a discontinuous system designed for DNA separation. Quantities of 0.5 mol/L Tris and 1.5 g Tris are dissolved in distilled H₂O. The volume is made up to 25 mL. A second sample solution is also prepared, containing 0.1 mol/L EDTA and 1.0 g EDTA sodium salt. The ingredients are also dissolved in distilled H₂O and made up to a volume of 25 mL.

The sample buffer (total volume 25 mL) is prepared in the following way: 500 μL of 0.5 mol/L Tris solution (final concentration 10 Mmol/L) is mixed with 250 μL 0.1 mol/L EDTA (final concentration 1 Mmol/L, 10 mg of bromophenol blue and 23 mL distilled H₂O. An additional quantity of 1.25 mL of distilled H₂O is added.

The solution is thoroughly mixed and the pH was adjusted with acetic acid to pH 7.5. 2 μL of sample buffer was then mixed with each 4 μL DNA sample, to make up the application volume to 6 uL.

The marker used is a 1:50 solution of Promega P. Gem Marker G147 and electrophoresis fluid.

The GenePhor Electrophoresis Unit (Pharmacia Biotech) is set at 15° C.

0.5 mL of Kerosene serving as insulating fluid is spread evenly onto the plate of the electrophoresis unit to continuously cool it.

The gel is positioned on the cooling plate with sample wells being on the cathode side. Air bubbles trapped under the gel are eliminated manually.

The buffer strips are placed in the slots provided, with their narrow base touching the plate.

Recommended running conditions are as follows: voltage 600V,

the current 25 mA and power 15 W for a duration of 80 mins at 15° C. However, better results may be obtained with a lower voltage. At 300V the gel may not dry out as quickly, giving by virtue of a longer run, a superior final picture.

Once electrophoresis is completed, the gel is stained by silver staining.

All procedures are based on all reagents used at an ambient temperature of +20° C. to +27° C. 4 aliquots of reagents (125 mL each) are prepared.

A Fixing solution, (containing Benzene sulphonic acid at 3.0% w/v in 24% v/v ethanol) is prepared by mixing 25 mL of a Fixing solution 5× and 100 mL 24% ethanol.

A Staining solution (containing Silver nitrate; 1.0 w/v, Benzene sulphonic acid at 0.35% w/v) is prepared by mixing 25 mL of Staining solution 5× and 100 mL of distilled H₂O.

The Developing solution concentrate (5×, containing Sodium carbonate at 12.5% w/v) is prepared by mixing 25 mL Sodium carbonate 5×, 125 μL of Sodium thiosulphate (2% w/v in H₂O), 125 μL of 37% Formaldehyde/distilled H₂O and 100 mL distilled H₂O.

A Stopping and preserving solution (containing Acetic acid at 5% v/v, Sodium acetate at 25% w/v and Glycerol at 50% v/v) is prepared by mixing 25 mL of Stopping and preserving solution 5× and 100 mL of distilled H₂O.

It should be noted that the Developing solution with Formaldehyde is unstable and is prepared immediately before use.

The gel is soaked in the Fixing solution and placed onto an orbital shaker for a minimum time of 30 minutes. The gel is then incubated in the staining solution for a further 30 minutes and washed in a quantity of distilled H₂O for a period of 1 minute.

The water is then poured off and the gel is placed into the Developing solution for a period of 6 minutes.

The DNA bands become visible. The gel is then soaked in the Stopping and Preserving solution for a minimum period of 30 mins (can be left overnight). The gel is retrieved and dried.

The gels may then be photographed for future analysis, and the photographs visually enhanced,

Only those DNA bands that are clearly visible are transferred onto graph paper. Each band is given an identity (A1, A2, etc.) at this stage. DNA bands which are not distinct, (faint bands), are discarded. DNA bands appearing on the series of gels where identical primers are used are carefully compared.

Size Estimation of RAPD or ISSR Generated DNA Bands

To estimate the size of DNA bands of each species of woody perennial, the following terms and equations are used.

x=log size

y=log distance

a=intercept of slope with y axis (additive constant)

b=slope (multiplicative constant)

x=(a−y)·b (the formula for slope).

Then
y=a−x/b
x1=(a−y1)·b
x2=(a−y2)·b
(x1−x2)=(y2−y1)·b
Consequently
b=(x1−x2)/(y2−y1)
a=(x/b)+y=(x1/b)+y1=(x2/b)+y2
Where
xu=log size of an unknown band
yu=measurement of distance of an unknown band.
Then
x=(a−yu)·b
To calculate antilog of xu (in band size)=exp·(pertinent coordinates).
To calculate the initial x value x=LN (K9)
Cladistic Analysis of Morphological Characters
Genetic Distances

Pairwise genetic distances are calculated on the basis of the proportion of fragments, using formula 1−2N_{xy}/(Nₓ+N_{y}) where N_{xy} is the number of bands shared by specimens x and y, and Nₓ is the number of bands from specimen x (based on Upholt 1977, as cited by Avise 1993). The resulting values, provided as a percentage, then provide an indication of the relatedness between species. If two species or too distantly related, hybridisation is less likely to be successful. Also, if two species are too closely related, hybridisation is less likely to be successful: if two species are very closely related, there is a greater likelihood that the two species have originated from the same region and from a common ancestor. In the process of speculation, barriers to hybridisation, such as genetic incompatibilities may arise, allowing for the two distinct species to co-exist. Although exception may be expected, generally, a relatedness of between about 10% and about 90%, more typically a relatedness of between about 15% and about 80%, even more typically a relatedness of between about 20% and about 70%, even more typically a relatedness of between about 20% and about 60%, even more typically a relatedness of between of about 20% and 50%, would indicate a reasonable likelihood of successful hybridisation.

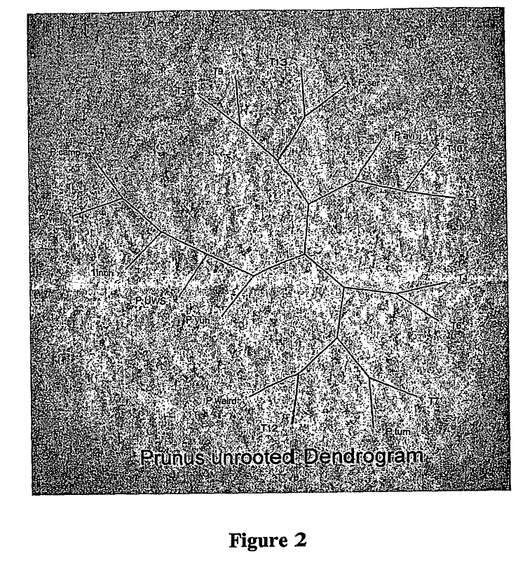

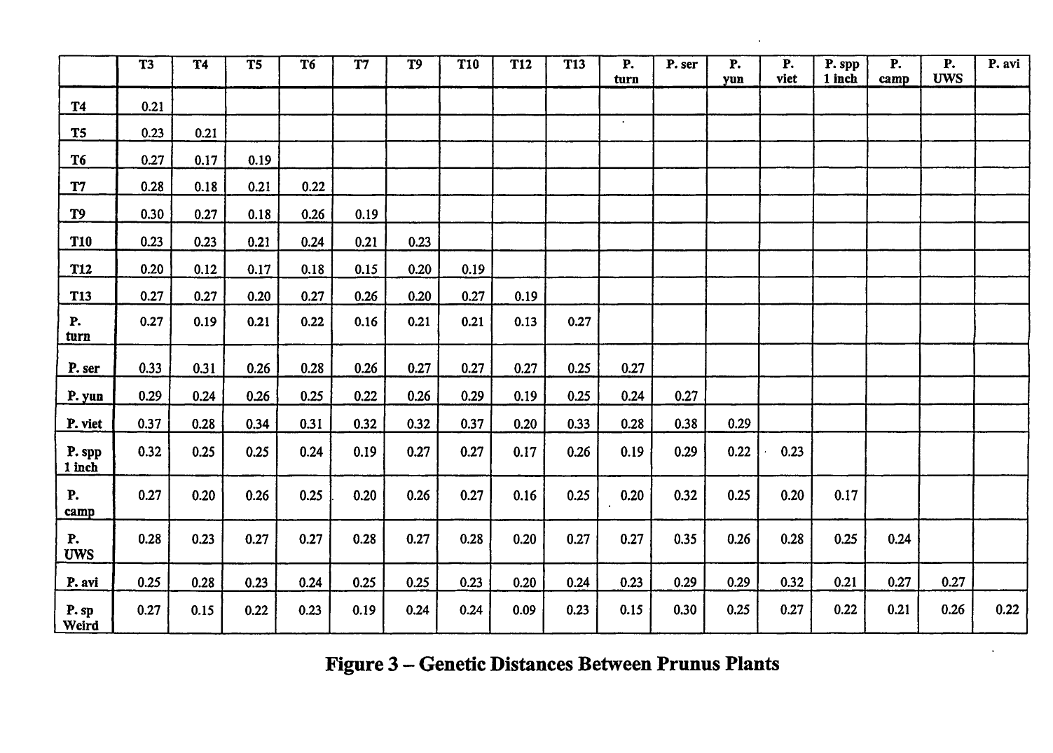

A table of genetic distances for the cherries listed in the cladogram of FIG. 1 and the dendrogram of FIG. 2 is provided in FIG. 3.

Improved Method for Increasing the Ploidy of Woody Perennial Plants

It has been found that exposure of plant tissue to relatively high concentrations of colchicine results in improved yield of cells in which chromosome multiplication has been effected. It has also been found that improved yields may be attained more reproducibly than is attained with known methods. In addition, the yield of cells having an increase in ploidy may be improved when exposure of the plant tissue to the agent capable of inhibiting spindle formation is commenced substantially coincidental with the breaking of dormancy of the plant tissue. That is, when the plant tissue or plant is put in an active state with respect to cell division.

Accordingly, the method of increasing ploidy in cells of a woody perennial plant comprises:

contacting plant tissue comprising dividing cells with an effective amount of a composition comprising an agent capable of inhibiting spindle formation, wherein said contacting commences substantially coincidental with breaking dormancy of said plant tissue.

The plant tissue may be at least one bud grafted onto a rootstock plant. The apical shoot and all buds of the rootstock plant may be removed, so as to allocate more of the plant resources to the grafted bud(s). The plant tissue may be a single grafted bud.

The plant tissue may be exposed to ultraviolet, or fluorescent light or to a mercury and/or sodium lamp substantially continuously subsequent to said contacting at least until growth from the treated tissue occurs.

The method may comprise the following steps:

contacting plant tissue comprising dividing cells with an effective amount of a composition comprising about 0.5% w/v to about 3% w/v colchicine.

The method of increasing ploidy in cells of a deciduous woody perennial plant may comprise:

contacting at least one bud of said plant, wherein said bud comprises actively dividing cells, with a composition comprising about 0.5% w/v colchicine to about 3% w/v colchicine,

at least partially enveloping said bud with a material capable of inhibiting gaseous exchange, wherein said contacting is substantially continuous over a period of from about 5 days to about 15 days.

The method be used for generating a plant having a desired ploidy level, the method comprising:

contacting plant tissue comprising dividing cells with an effective amount of a composition comprising about 0.5% w/v colchicine to about 3% w/v colchicine,

generating at least one plant from tissue so contacted, and

selecting at least one plant having the desired ploidy level.

The above methods may be used in a method of generating a plant, the method comprising:

contacting plant tissue comprising dividing cells with an effective amount of a composition comprising about 0.5% w/v colchicine to about 3% w/v colchicine,

selecting plant tissue of increased ploidy level,

generating at least one plant from said selected plant tissue, and

crossing said generated plant with a plant of the same or different ploidy level.

The above methods may be used in a method of generating a plant having at least one desired trait, the method comprising:

contacting plant tissue comprising dividing cells with an effective amount of a composition comprising about 0.5% w/v colchicine to about 3% w/v colchicine,

selecting plant tissue of increased ploidy level,

generating at least one plant from said selected plant tissue,

crossing said generated plant with a plant of the same or different ploidy level, and

selecting at least one progeny plant having the desired trait.

The above methods may be used in a method of generating a plant having at least one desired trait, the method comprising:

contacting parental diploid plant tissue comprising dividing cells with an effective amount of a composition comprising about 0.5% w/v colchicine to about 3% w/v colchicine,

selecting tetraploid tissue from said treated plant tissue,

generating at least one tetraploid plant from said tetraploid tissue,

crossing said tetraploid plant with a diploid plant, and

selecting at least one progeny plant having the desired trait.

The agent capable of inhibiting spindle formation in the plant tissue may be any suitable agent, for example colchicine, oryzalin (Surflan™), trifluralin, amiprophos-methyl, and N₂O gas. It is also envisaged that a combination of agents may be used.

Where the agent capable of inhibiting spindle formation is colchicine, the colchicine may be administered as a composition comprising about 0.5% w/v colchicine to about 3% w/v colchicine. Thus, the composition may comprise colchicine in a concentration of about 0.5% w/v, about 0.6% w/v, about 0.7% w/v, about 0.8% w/v, about 0.9% w/v, about 1% w/v, about 1.1% w/v, about 1.2% w/v, about 1.3% w/v, about 1.4% w/v, about 1.5% w/v, about 1.6% w/v, about 1.7% w/v, about 1.8% w/v, about 1.9% w/v, about 2% w/v, about 2.1% w/v, about 2.2% w/v, about 2.3% w/v, about 2.4% w/v, about 2.5% w/v, about 2.6% w/v, about 2.7% w/v, about 2.8% w/v, about 2.9% w/v or about 3% w/v.

Where the agent capable of inhibiting spindle formation is oryzalin, the oryzalin may be administered as a composition comprising about 0.001% w/v oryzalin, about 0.005% w/v oryzalin, about 0.01% oryzalin, about 0.05% w/v oryzalin, 0.1% w/v oryzalin, or about 0.5% w/v oryzalin.

Contact of the plant tissue with the agent, for example colchicine, may be commenced substantially coincidental with, or soon after the plant tissue has broken dormancy. This may be referred to as priming the plant or plant tissue before, or substantially coincidental with, contact with the agent, such that the growing point is contacted substantially at the earliest time of activity. For example, where the plant tissue is one or more buds on a rootstock, the rootstock may be exposed to conditions sufficient to break dormancy prior to contact with the composition. Conditions sufficient to break dormancy will depend on the particular plant and may be determined by methods known to those of skill in the art. For example, a plant having a particular chill requirement may be maintained at an appropriate temperature for a time sufficient to satisfy the chill requirement and then exposing the plant to an appropriate (warmer) temperature for a time sufficient to prime bud break. For example, Prunus salicina low chill 2N=16, chill at 5° C. for 200 hours; Prunus domestica (2N=48), chill at 5° C. for 1200 hours, then hold at 22° C. to break dormancy.

Similarly, it is envisaged that the method is also applicable to increasing the ploidy of cells in grafted plant tissue. The grafted tissue or scion may have different requirements for breaking dormancy compared with the rootstock, for example the rootstock may have a lower chill requirement than does the scion or the rootstock may have a higher chill requirement than the scion.

One or more grafted buds may be used. Removal of all buds and/or growing shoots from the rootstock, before or once the bud(s) have taken, will ensure that an increased proportion of the plant resources will be directed towards the grafted bud(s), increasing the likelihood of cells surviving the treatment and mutating. Use of a single grafted bud on the rootstock, which will therefore be ‘apically dominant’ will further increase the chances of survival and mutation of grafted cells. In order to reduce the amount of time required to break dormancy of the plant, bud breaking agents such as hydrogen cyanimide may be employed or treatment such as exposure to ultraviolet or fluorescent light or mercury and/or sodium vapour lamp(s) may be used.

Exposure of treated plant tissue to ultraviolet or fluorescent light or mercury and/or sodium vapour lamp(s) substantially continuously during and/or subsequent to treatment with colchicine (that is, not allowing a nocturnal phase) may also be carried out so as to encourage growth, cell division and therefore increase the likelihood of successful mutation of the plant cells. Ideally, although not necessarily, exposure is carried out at least until growth from the treated tissue occurs.

Contacting the plant tissue with colchicine, or any other suitable agent capable of inhibiting spindle formation, may be effected by any suitable means, such as by substantially immersing or substantially submersing the plant tissue into the composition, for example by dipping the plant tissue into the composition, or by dripping or dropping the composition onto the plant tissue, for example by use of a pipette, dropper or syringe, or by spraying the composition onto the plant tissue, or by painting the plant tissue with the composition, such as by an appropriately sized paintbrush, cloth or cotton bud. The composition may also be administered to the plant tissue by injection, for example by use of a hypodermic-type syringe.

The composition may be administered to the plant tissue before or after the plant tissue has been at least partially enveloped in an absorbant material. The absorbant material may be any suitable absorbant material, for example, the absorbant material may be laboratory standard cotton or cotton wool, sponge, foam. For example, the plant tissue may be at least partially enveloped in an absorbant material and then the composition administered by any of the above-described means such that the absorbent material becomes at least partially saturated with the composition. The composition may be administered such that the absorbant material becomes saturated with the composition. Administration of the composition to the absorbant material may be describer as indirect administration of the composition to the plant tissue. Administration of the composition to the plant tissue may be indirect or direct administration.

The composition may be in any suitable form. For example, the composition may be in the form of a solution, paste, or salve. The composition may be in the form of an aqueous solution.

After administration of the composition to the plant tissue, the plant tissue, which may or may not be at least partially enveloped in an absorbant material, may be at least partially enveloped with a material capable of inhibiting gaseous exchange. The material capable of inhibiting gaseous exchange may be capable of partially, substantially completely or completely inhibiting gaseous exchange. For example, the material capable of inhibiting gaseous exchange may be a plastic film, for example in the form of a bag. Thus, for example, the plant tissue may be at least partially enveloped in an absorbant material, to which the composition comprising the agent capable of inhibiting spindle formation, such as colchicine, is administered in an amount sufficient to at least partially or completely saturate the absorbant material, before the absorbant material is at least partially enveloped in a plastic film or bag.

The agent capable of inhibiting spindle formation may be administered in combination with at least one additional agent capable of enhancing penetration of the spindle formation inhibiting agent into the plant tissue. These additional agents may collectively or individually be referred to, for the purposes of the present invention, as a carrier(s). Suitable carriers include, for example, surfactants, wetting agents, oils and dimethylsulfoxide. The oil may be, for example a non-phytotoxic oil or a phytotoxic oil used in a non-toxic amount. Where a carrier is used a lower concentration of the agent capable of inhibiting spindle formation, such as colchicine, may be used compared to the absence of a carrier. A combination of different types of carrier(s) may also be used.

The carrier(s) may be administered simultaneously with the agent capable of inhibiting spindle formation, such as by contacting the plant tissue with a composition comprising an agent capable of inhibiting spindle formation and one or more carriers, or by sequential administration of the carrier(s) and the agent capable of inhibiting spindle formation. When administered sequentially, the carrier(s) and the agent capable of inhibiting spindle formation may be administered to the plant tissue in any order, for example administration of the carrier(s) to the plant tissue prior to administration of the agent capable of inhibiting spindle formation or by administration of the agent capable of inhibiting spindle formation to the plant tissue prior to administration of the carrier(s). When administered sequentially, the carrier(s) and the agent capable of inhibiting spindle formation are administered over a time period which provides for overlapping effect.

Substantially continuous contact of the plant tissue with the agent may be achieved by a single administration of the composition or by multiple administrations of the composition to the plant tissue. In this manner the concentration of the agent capable of inhibiting spindle formation may be maintained at or near an optimum level. For example, fresh applications of the composition may be administered one, two, three, four or more times per day for the period of contact of the plant tissue with the agent. Where multiple administrations of the composition are undertaken, the absorbant material at least partially enveloping the plant tissue may or may not be removed and may or may not be replaced as part of the multiple administration(s).

The plant tissue subject to the method of the invention may be maintained under conditions which optimise cell division or growth. Where the plant tissue subject to the method of the invention is maintained under conditions of a naturally-occurring diurnal cycle, which conditions may be natural or artificially induced or simulated, at least one of the administrations of the composition may be administered at a time in the diurnal cycle when cell division is relatively high. For example, at least one administration of the composition may occur early in the morning. Where multiple administrations occur over two or more days, at least one of each administration on each day may occur early in the morning.

Where the plant tissue is maintained under continuous lighting to encourage continuously elevated cell division, administration of the composition may be evenly spaced, for example once every 2, 4, 6, 8, 10, 12, 14, 16, 18, 20, 22 or 24 hours.

In the method of the invention the plant tissue to which the agent capable of inhibiting spindle formation is applied, which may be the original growing point of the plant, may be killed during contact with the agent. Apical side buds may be produced adjacent to the killed main bud. The apical side buds may be mutated, such as by increased ploidy compared to the original plant tissue.

Plant tissue may be assessed for increase in ploidy by any suitable method known to those skilled in the art. For example, increase in ploidy may result in increased cell size that brings about thicker, broader leaves and larger flowers and fruit, shoots of plants having increased ploidy may be thicker and may have shortened internodes and wider crotch angles. As further examples, polyploidy may be evidenced by larger pollen size, or by greater number of chloroplasts per guard cell, or by larger guard cells and stomates. Depending on the type of tissue being analysed for increase in ploidy, methods including root tip squashes, pollen mother cell squashes, pollen grain size and germinal pore counts, stomata size and density determination, and gross morphology may also be used. Appropriate methods for chromosome staining and counting are also known in the art. Advanced techniques such as measurement of the nuclear DNA content of the plant cells, such as by flow cytometry, or microspectrophotometry may be used for ploidy determination.

The method of the invention is suitable for increasing the ploidy of numerous plant species. For example, the method may be applied to any woody perennial plant. The woody perennial plant may be deciduous or evergreen. Examples of deciduous woody perennial plants to which the method may be applied are plants of the genus Prunus. Plants of the genus Prunus to which the method of the invention may be applied include, for example, P. mira, P. mandschurica, P. ansu, P. davidiana, P. brigantiaca, P. ceracifera, P. mume, P. domestica, P. salicina, P. armeniaca, P. simonii, P. americana, P. sibirica, P. mexicana, P. hortulana, P. angustifolia, P. munsoniana, P. umbellata, P. communis, P. persica, P. persica var nectarina, P. pumila, P. besseyi, P. humilis, P. ceracoides, P. avium, P. pseudocerasus, P. campanulata. Where such plants may be useful in a breeding program but have varying chromosome numbers, the method of the invention may be used to increase, for example to double, the chromosome number. For example, Prunus salicina (2N=16); Prunus avium (2N=16); Prunus pseudocerasus (2N=32). For example, the method of the invention may be used to generate tetraploid tissue or a tetraploid plant from Prunus avium which may then be crossed with Prunus pseudocerasus. For example, the method may be used in assisting a breeding program of crossing a European sweet cherry with 16 chromosome pairs with another species with 32 chromosome pairs. Examples of other genera to which the method of the invention may be applied include Pyrus (pear), such as P. pyrifolia and P. communis and Malus (apple), such as M. domestica, M. asiatica, and M. formosana and citrus, such as C. medica, C. limonia, C. sinensis, C. grandis, C. paradisii, C. ichangensis, C. aurantifolia, C mitis, C. nobilis, Poncirus trifoliata, Persea (avocado), Mangifera (mango), Punica (pomegranate), Olea (olive).

The ability to improve the generation of plant tissue having increased ploidy levels, that is, the ability to improve the generation of polyploid plant tissue and therefore plants, may permit the skilled person to more reliably address issues associated with plant breeding and the generation of new plant species and varieties having desirable characteristics. For example, differences in ploidy levels or chromosome number in prospective parental plants constitutes a difficulty in generating progeny, which may be substantially overcome by manipulating the ploidy levels of the prospective parental plant(s) prior to hybridisation. Manipulation of the ploidy level or chromosome number may or may not equalise the ploidy level or the chromosome number of the prospective parents in order to substantially overcome difficulty in generating progeny. For example, a pollen mixture or polymix may be used to improve the likelihood of successful hybridisation where differences in the ploidy level or chromosome number remain after the method of the invention. Alternatively, the method may be used to restore fertility in a plant variety or cultivar having desired traits. For example, the plant variety having desired traits may be the product of hybridisation between plants of a different species or genera and, due to the failure of the chromosomes to pair correctly in meiosis, will often be sterile. Restoration of fertility in such a variety may be accomplished by doubling the chromosome number. As a further example, it may be desirable to create sterile cultivars of a species, such as in the situation where it is desirable to limit the ability of an important agricultural, commercial or nursery species or variety to reproduce and spread. For example, doubling the chromosome number of a plant may result in sterility due to multiple homologous chromosomes and resultant complications in meiosis. Alternatively, or in addition, sterile triploid plants may be created by hybridisation of a tetraploid with a diploid. In commercial applications where the plant variety or species is a fruiting plant, this highlights a further use to which the method of the invention may be applied, that being, the generation of seedless (or substantially seedless) fruit. The method may also find use in the development of plant varieties having enhanced pest resistance and stress tolerance. For example, increasing the chromosome number and related gene dose has been known to enhance the expression and concentration of secondary metabolites and defence chemicals of the plant.

The use of pollen mixtures obtained from a number of woody perennial varieties, species, or genera may be used to increase the likelihood of successful hybridisation, particularly where the seed parent and the pollen parent are of different species or incompatibility groups, such as occur in cherries (Prunus avium, and related species). Use of pollen mixtures may also be used in overcoming self-incompatibility such as occurs in, for example, plums (for example Prunus salicina and P. domestica), apricots (P. armeniaca) and almonds (P. amygdalus).

The skilled addressee will be aware that there are a number of possibilities for use of the treated plant tissue. For example, the directly treated tissue or apical side buds may be permitted to develop to a stage where they can be assessed for increase in ploidy. The buds may be permitted to continue to develop in situ or one or more buds may be excised and engrafted to one or more alternative rootstock(s). The buds, either in situ as treated or engrafted, may be permitted to develop to maturity, for example to flowering or fruiting stage. The method of the invention thus provides a method for the generation of new plant varieties, cultivars and breeding lines.

As described above, the method of the invention may also be used in the production of a substantially seedless plant variety. For example, this is advantageous in the production of commercially important fruit crops, such as stone fruit or citrus, avocado, mango, or olive. For example, plant tissue of a diploid parental plant having one or more desirable characteristics, such as flesh colour, sugar levels, skin colour, acidity, disease resistance, fruit size, maturity time may be subjected to the method of the invention and resultant tetraploid plant tissue selected. The tetraploid plant tissue is allowed to develop to maturity, either in situ or after excision and engrafting, and may then be hybridised or backerossed with the original diploid parent plant. The triploid progeny will be substantially seedless. The original parental plant may be either polyembryonic or monoembryonic.

The present invention will now be described, by way of example only, with reference to the following examples, which are not to be taken to be limiting to the scope or spirit of the invention in any way.

### EXAMPLES

### Example 1

The incorporation of the male sterility gene into a breeding line of a woody perennial species may be achieved by the following steps:
Identification and collection of a broad range of germplasm of potential interest, i.e. with one or more desirable agronomic characteristics. This germplasm can include both that which is in the public domain as well as wild species.
For phenotypic characteristics, the identification and of desirable characteristics can be done by visual observation of shape, pubescence, suture, ripening sequence, oxidation, pit adhesion, colour of the shin, flesh and pit cavity, etc.
For non-phenotypic characteristics, i.e. the levels of total soluble solids, firmness, the identification and of desirable characteristics can be done by use of standard apparatuses such as a refractometer and penetrometer respectively.
From within this genome, identify varieties that has the male sterility as part of its genome. It is most easily found by observing those plants that do not produce viable pollen, these flowers have anthers that are white in colour.
Establish a field trial by planting out a given number of plants from within the collection and allow them to self pollinate to determine whether and for which desirable characteristic(s) a plant or several plants are homozygous for the characteristic(s) under study. This is established by knowing the dominance relationship of each desirable trait.
Utilising the process of “artificial emasculation” and normal cross breeding techniques as described above, incorporate the male sterility gene into one or more varieties that carry the best combination of desirable characteristics.
Allow the varieties with the male sterility gene to self pollinate and undertake progeny testing to identify those that are homozygous for the male sterility gene, and which have the best combination of other desirable characteristics.
Once several of such varieties have been created, a breeding line is established that can progressively be refined using the following critical steps in the process.

A schematic/flow chart in respect of a simple method according to the invention is provided in FIG. 3.

### Example 2

The breeding lines obtained according to the method of example 1 can be employed to generate new woody perennial varieties by the following steps:
Identify a trial block of land that is physically isolated from other trees with which the species under consideration could otherwise cross pollinate. This distance should be sufficiently large to stop bees and any other natural vectors from travelling between the plots.
Choose the best homozygous lines for particularly desired commercial traits in a fruit under consideration, and which do NOT carry the male sterility gene.
Interplant these fruit plants in a block where each plant is alternated with a plant breeding line incorporating a male sterility gene (and the other desired characteristics comparable to those available in the above mentioned homozygous lines) (see Table 2, next page).
Utilise natural vectors ie bees and other common pollinators, to carry pollen from the non-male sterile trees onto the male sterile trees. (However, pollination may be conducted by hand if so desired).
It is not necessary to use a mixture of plants (i.e. “A”, . . . “G”) among the plants that carry the male sterility gene (i.e. “MS”). Mono-cultures (i.e. all “A” or all “D”, etc) can also be used to increase the specificity of the crosses.
Once this process has been completed, the progenies are harvested (from the male sterile plants only) and the kernels extracted for evaluation.
The kernels thus harvested and pre-germinated in refrigerated conditions under moist stratification so as to shorten the juvenile period to allow for earlier evaluation—which is as short as only 2 years maximum.
The set of controlled cross-bred progenies so obtained is then planted out under high density in the field and evaluated for certain desired commercial characteristics as nominated below.
For the process described above to occur efficiently, the plants should flower simultaneously. This is not always the case. In these circumstances, one of two somewhat more manual process can be used:
   pollen from a from previous seasons is stored by freezing, and applied by hand onto the male sterile trees, or

flowering wood of one or more of the selections may be harvested and put through its chill requirement in a cool room and then taken out and forced under higher temperatures to flower. Then:

TABLE 2
planting pattern (Note: MS = Male Sterile Plants; “A” etc = other
homozygous breeding line)

| | | |
|---|---|---|
| MS | “C” | MS |
| “A” | MS | “F” |
| MS | “D” | MS |
| “B” | MS | “G” |
| MS | “E” | MS |

pollen may be collected and applied manually as described above, or the flowering wood is hung among the trees that carry the male sterility gene, and allow natural vectors to “do their thing”

Those plants with the desired attributes are evaluated for commercial potential, using the visual and other methodologies outlined above.

Some examples of the use of this method in accurate, (non manual), hybridisation include the following:

1. Incorporation of low chill characteristics to provide early fruiting

2. Long fruit development period characteristics (or short fruit development)

3. Sub-acidness (which is a single gene for the deletion of acid in peach and nectarine

4. Control of fruit shape, fruit texture, total soluble solids (sugars).

5. Control of skin pubescence (and length of hair) and skin colour.

6. Pigmentation, stone adhesion to fruit fresh.

### Example 3

This example demonstrates plantings in a polycross trial. The trial plot was designed to exploit male sterility and low chill requirement previously incorporated into the Peach 82-12 and the nectarine 82-25N, these were used as female parents and allowed to cross with the surrounding varieties according to the orchard layout provided in Table 3 below.

TABLE 3
Planting pattern: polycross trial performed at Nambucca, Australia, 1989

| | VARIETY | ROOTSTOCK | PLANTED | COMMENTS |
|---|---|---|---|---|
| ROW 1 | | | | |
| 1 | FLA 4-3 | Coastal Peach | September 1992 | |
| 2 | FLA 8-1 | Coastal Peach | October 1989 | |
| 3 | FLA 84-16N | Coastal Peach | September 1992 | |
| 4 | FLA 8-6 | Coastal Peach | October 1989 | |
| 5 | FLA 82-17 | Coastal Peach | August 1993 | |
| 6 | FLA 8-13N | Coastal Peach | October 1989 | |
| 7 | FLA 82-25 N | Coastal Peach | October 1989 | Male Sterile Nectarine |
| 8 | FLA 9-11 | Coastal Peach | October 1989 | |
| 9 | SHANGHAI | Own Roots | October 1989 | |
| | SEEDLING | | | |
| 10 | FLA 82-3 | Coastal Peach | October 1989 | |
| 11 | FLA 82-25N | Coastal Peach | October 1989 | Male Sterile Nectarine |
| 12 | FLA 82-9W | Coastal Peach | October 1989 | |
| 13 | FLA 82-12 | Coastal Peach | October 1989 | Male Sterile Peach |
| 14 | M 3-6 | Coastal Peach | September 1992 | |
| 15 | M 3-5 | Coastal Peach | September 1992 | |
| 16 | FLA 82-24 | Coastal Peach | October 1989 | |
| 17 | FLA 82-25N | Coastal Peach | October 1989 | Male Sterile Nectarine |
| 18 | FLORDABELL | Coastal Peach | October 1989 | |
| 19 | FLA 82-12 | Coastal Peach | October 1989 | Male Sterile Peach |
| 20 | M2-4N | Coastal Peach | September 1992 | |
| 21 | ROOTSTOCK | Own Roots | October 1989 | |
| 22 | SUNFIRE | Coastal Peach | October 1989 | |
| 23 | FLA 82-25 N | Coastal Peach | October 1989 | Male Sterile Nectarine |
| 24 | FLA 82-24 W | Coastal Peach | October 1989 | |
| 25 | FLA 82-12 | Coastal Peach | October 1989 | Male Sterile Peach |
| 26 | FLA 82-9W | Coastal Peach | October 1989 | |
| 27 | ROOTSTOCK | Own Roots | October 1989 | |
| 28 | FLA 9-14 | Coastal Peach | October 1989 | |
| Row 2 | | | | |
| 1 | 9-20 C | Coastal Peach | | |
| 2 | Shanghai seedling | Own Roots | | |
| 3 | Shanghai seedling | Own Roots | | |
| 4 | FLA 82-12 | Coastal Peach | October 1989 | Male Sterile Peach |
| 5 | FLA 86-28C | Coastal Peach | September 1992 | |
| 6 | FLA 86-28C | Coastal Peach | September 1992 | |
| 7 | FLA 86-28C | Coastal Peach | September 1992 | |
| 8 | Coastal Peach | Coastal Peach | October 1989 | |
| 9 | FLA 82-12 | Coastal Peach | | Male Sterile Peach |
| 10 | FLA 84-12C | Coastal Peach | October 1992 | |
| 11 | FLA 82-25N | Coastal Peach | October 1989 | Male Sterile Nectarine |
| 12 | FLA 86-28C | Coastal Peach | September 1992 | |
| 13 | M 3-8 | Coastal Peach | September 1992 | |
| 14 | M 3-7 | Coastal Peach | September 1992 | |
| 15 | M 3-6 | Coastal Peach | September 1992 | |
| 16 | Sunbob | Coastal Peach | September 1992 | |
| 17 | FLA 82-25N | Coastal Peach | October 1989 | Male Sterile Nectarine |
| 18 | M 3-8 | | February 1992 | |
| 19 | M 3-7 | | February 1992 | |
| 20 | M 3-6 | | February 1992 | |
| 21 | FLA 82-12 | Coastal Peach | October 1989 | Male Sterile Peach |
| 22 | FLA 84-22 | Coastal Peach | September 1992 | |
| 23 | FLA 82-25N | Coastal Peach | October 1989 | Male Sterile Nectarine |
| 24 | | | | |
| 25 | | | | |
| 26 | FLA 4-3 | | September 1992 | |
| 27 | V.V 91-1 | Own Roots | October 1989 | |

The low chill and male sterility genes were incorporated into an F₁ progeny which has been used continuously to generate new peach and nectarine cultivars and to generate new trait-targeted MS breeding lines. Pollen polymixes obtained from a range of selected commercial varieties have been continuously used for crossing to the to broaden the gene base. For example, in 1999 there were six lots of pollen polymixes used in crosses.

### Example 4

This example demonstrates plantings in another polycross trial performed at Nambucca, Australia in 1992. The trial plot was designed to exploit male sterility and low chill requirement previously incorporated into the male sterile Peach 82-12 and the male sterile nectarine 82-25N, these were used as female parents and allowed to cross with the surrounding varieties, in two rows according to the orchard layout provided in Tables 4A and 4B below.

The resulting F₁ generation plants were then observed for desired traits and selected based on the following fruit and chill requirement criteria: round with no point on end; no suture bulge; >60% blush; >10 Brix; no red pigment around pit; chill requirement <400 hours. The selected F₁ plants for further development of male sterile breeding lines are shown in Table 5.

All selections also had an upright tree habit, long lanceolate leaves, pink flowers, melting acid flesh and all were heterozygous male fertile.

The hybrids shown in Table 5 can then be used for back-crosses either by self-fertilisation, cross-fertilisation with polymixes of pollen from all, or a selection of the hybrids, or cross-fertilisation with one or more selected varieties which are at least heterozygous for the same allele for male sterility, or any combination of such crosses so as to ultimately obtain one or more male sterile breeding lines which are also preferably homozygous for one or more desirable horticultural traits, such as, for example, those listed above or in Tables 1, 4 or 5. Preferably a range of male sterile breeding lines will be obtained with different combinations of traits (preferably being homozygous for each of these), to enable directed ‘insertion’ of one or more desired traits into horticultural varieties lacking those one or more traits without affecting other desired phenotypic traits of those varieties.

TABLE 4A
Planting pattern: polycross trial - Plantation Row 1

| | | | | | Flesh | | | | | | | | | |
|---|---|---|---|---|---|---|---|---|---|---|---|---|---|---|
| | | | | | Firmness | | | | | | | Color/ | | |
| | | | | | Melting/ | | | | Flow- | | | Red | | |
| Parent | | Pubes- | Blush | Flesh | Non | | Fruit | Leaf | er | | Stone | round | Flesh | |
| Variety | Chill† | cence | (%) | Color | Melting | Brix | Shape | Color | Color | Suture | Adhesion | the pit | Acidity | Fertility |
| Fla 4-3 | 350 | Yes | 40 | Yellow | Melting | 10 | Pointed | Light | Pink | 6 | Cling | Yes | Acid | Fertile |
| | | | | | | | | Green | | | | | | |
| Fla 8-1 | 200 | Yes | 60 | Yellow | Melting | 10\11 | Round | Light | Pink | 9 | Cling | No | Acid | Fertile |
| | | | | | | | | Green | | | | | | |
| Fla 84-16N | 300 | No | 80 | Yellow | Melting | 10\11 | Elongate | Light | Pink | 8 | Cling | Slight | Acid | Fertile |
| | | | | | | | | Green | | | | | | |
| Fla 8-6 | 250 | Yes | 70 | Yellow | Melting | 10\11 | Round/ | Light | Pink | 10 | Cling | Slight | Acid | Fertile |
| | | | | | | | Excellent | Green | | | | | | |
| Fla 8-13N | 300 | No | 60 | Yellow | Melting | 10\12 | Very | Light | Pink | 7 | Cling | No | Acid | Fertile |
| | | | | | | | Pointed | Green | | | | | | |
| Fla 82-25N | 200 A | No | 80 | Yellow | Melting | 10\11 | Round | Light | Pink | 10 | Semi Free | No | Acid | Male |
| | | | | | | | | Green | | | | | | Sterile |
| Fla 9-11 | 300 | Yes | 70 | Yellow | Melting | 10\11 | Round | Light | Pink | 9 | Semi Free | No | Acid | Male |
| | | | | | | | | Green | | | | | | Sterile |
| Shanghai | 200 | Yes | 40 | White | Melting | 11\12 | Pointed | Dark | Pink | 5 | Freestone | Yes | Acid | Fertile |
| | | | | | | | | Green | | | | | | |
| Fla 82-3 | 300 | Yes | 60 | Yellow | Melting | 10\12 | Ovoid | Light | Pink | 7 | Cling | No | Acid | Fertile |
| | | | | | | | | Green | | | | | | |
| Fla 82-25N | 200 | No | 80 | Yellow | Melting | 10\11 | Round | Light | Pink | 10 | Semi Free | No | Acid | Male |
| | | | | | | | | Green | | | | | | Sterile |
| Fla 82-9W | 200 | Yes | 60-70 | White | Melting | 10\11 | Round | Dark | Pink | Nil 10 | Cling | No | Acid | Fertile |
| | | | | | | | | Green | | | | | | |
| Fla 82-12 | 200 | Yes | 70 | Yellow | Melting | 10 | Pointed | Light | Pink | 7 | Cling | No | Acid | Male |
| | | | | | | | | Green | | | | | | Sterile |
| M 3-6 | 350 | Yes | 70 | White | Melting | 10\11 | Round | Light | Pink | 9 | Cling | No | Acid | Fertile |
| | | | | | | | | Green | | | | | | |
| M 3-5 | 350 | Yes | 70 | Yellow | Melting | 10\11 | Round | Light | Pink | 9 | Cling | No | Acid | Fertile |
| | | | | | | | | Green | | | | | | |
| Fla 82-24W | 200 | Yes | 70-80 | Yellow | Melting | 11\12 | Round | Dark | Pink | 10 | Freestone | No | Acid | Fertile |
| | | | | | (9/10) | | | Green | | | | | | |
| Fla 82-25N | 200 | No | 80 | Yellow | Melting | 10\11 | Round | Light | Pink | 10 | Semi Free | No | Acid | Male |
| | | | | | | | | Green | | | | | | Sterile |
| New | A | Yes (8) | 50-60 | White | Melting | 10\12 | Round | Light | Pink | 9 | Freestone | No | Acid | Fertile |
| Flordabelle | | | | | | | | Green | | | | | | |
| Fla 82-12 | 200 | Yes | 70 | Yellow | Melting | 10 | Pointed | Light | Pink | 7 | Cling | No | Acid | Male |
| | | | | | | | | Green | | | | | | Sterile |
| M 2-4N | 350 | No | 70 | Yellow | Melting | 10\12 | Elongate | Light | Pink | 9* | Cling | No | Acid | Fertile |
| | | | | | | | | Green | | | | | | |
| Sunfree | 350 | No | 70 | Yellow | Melting | 10\12 | Elongate | Light | Pink | 8 | Cling | Some | Acid | Fertile |
| Nectarine | | | | | | | | Green | | | | | | |
| Fla 82-25N | 200 A | No | 80 | Yellow | Melting | 10\11 | Round | Light | Pink | 10 | Semi Free | No | Acid | Male |
| | | | | | | | | Green | | | | | | Sterile |
| Fla 82-24W | 300 | Yes | 70-80 | White | Melting | 11\12 | Round | Dark | Pink | 10 | Cling | No | Acid | Fertile |
| | | | | | | | | Green | | | | | | |
| Fla 82-12 | 200 | Yes | 70 | Yellow | Melting | 10 | Pointed | Light | Pink | 7 | Cling | No | Acid | Male |
| | | | | | | | | Green | | | | | | Sterile |
| Fla 82-9W | 200 B | Yes | 60-70 | White | Melting | 10\11 | Round | Dark | Pink | 10 | Semi | No | Acid | Fertile |
| | | | | | | | Root | Green | | | Cling | | | |
| Fla 9-14 | 250 | Yes | 70 | Yellow | Melting | 10\11 | Round | Light | Pink | 9 | Cling | No | Acid | Fertile |
| | | | | | | | | Green | | | | | | |

†Chill requirement - minimum hours required at less than 7° C. to break dormancy in 50% of the flower/leaf buds.

TABLE 4B
Planting pattern: polycross trial - Plantation Row 2

| | | | | | Flesh | | | | | | | | | |
|---|---|---|---|---|---|---|---|---|---|---|---|---|---|---|
| | | | | | Firmness | | | | | | | Color/ | | |
| | | | | | Melting/ | | | | | | | Red | | |
| Parent | | Pubes- | Blush | Flesh | Non | | | Leaf | Flower | | Stone | round | Flesh | |
| Variety | Chill† | cence | (%) | Color | Melting | Brix | Shape | Color | Color | Suture | Adhesion | the pit | Acidity | Fertility |
| Fla 9-20C | 300 | Yes | 30 | Yellow | Non Melting | 11 | Round | Light | Pink/ | Nil (10) | Cling | Yes | Acid | Fertile |
| | | (NN) | | | (mm) | | | Yellow | Non | | | | | |
| | | | | | | | | | Showy | | | | | |
| Shanghai | 200 | Yes | 40 | White | Melting | 11\12 | Pointed | Dark | Pink | 5 | Freestone | Yes | Acid | Fertile |
| | | | | | | | | Green | | | | | | |
| Shanghai | 200 | Yes | 40 | White | Melting | 11\12 | Pointed | Dark | Pink | 5 | Freestone | Yes | Acid | Fertile |
| | | | | | | | | Green | | | | | | |
| Fla 82-12 | 200 | Yes | 70 | Yellow | Melting | 10 | Pointed | Light | Pink | 7 | Cling | No | Acid | Male |
| | | | | | | | | Green | | | | | | Sterile |
| Fla 86-28C | 300 | Yes | 30 | Yellow | Non Melting | 10 | Round | Light | Pink/ | Nil (10) | Freestone | Yes | Acid | Fertile |
| | | (NN) | | | (mm) | | | Yellow | Non | | | | | |
| | | | | | | | | | Showy | | | | | |
| Fla 86-28C | 300 | Yes | 30 | Yellow | Non Melting | 10 | Round | Light | Pink/ | Nil (10) | Freestone | Yes | Acid | Fertile |
| | | (NN) | | | (mm) | | | Yellow | Non | | | | | |
| | | | | | | | | | Showy | | | | | |
| Fla 86-28C | 300 | Yes | 30 | Yellow | Non Melting | 10 | Round | Light | Pink/ | Nil (10) | Freestone | Yes | Acid | Fertile |
| | | (NN) | | | (mm) | | | Yellow | Non | | | | | |
| | | | | | | | | | Showy | | | | | |
| Coastal | 150 | Yes | 20 | White | Melting | 11 | Round | Dark | Pink | 9 | Freestone | Yes | Acid | Fertile |
| Peach | | (NN) | | | (MM) | | | Green | | | | | | |
| Fla 82-12 | 200 | Yes | 70 | Yellow | Melting | 10 | Pointed | Light | Pink | 7 | Cling | No | Acid | Male |
| | | | | | | | | Green | | | | | | Sterile |
| Fla 84-12C | 200 | Yes | Nil | Yellow | Non Melting | 10 | Round | Light | Pink/ | 10 | Cling | Yes | Acid | Fertile |
| | | (NN) | (0%) | | (mm) | | | Green | Non | | | | | |
| | | | | | | | | | Showy | | | | | |
| Fla 82-25N | 200 A | No | 80 | Yellow | Melting | 10\11 | Round | Light | Pink | 10 | Semi Free | No | Acid | Male |
| | | | | | | | | Green | | | | | | Sterile |
| Fla 86-28C | 300 | Yes | 30 | Yellow | Non Melting | 10 | Round | Light | Pink/ | Nil (10) | Freestone | Yes | Acid | Fertile |
| | | (NN) | | | (mm) | | | Yellow | Non | | | | | |
| | | | | | | | | | Showy | | | | | |
| M 3-8 | 350 | Yes | 60 | Yellow | Melting | 10\11 | Round | Light | Pink | 9 | Cling | Some | Acid | Fertile |
| | | | | | | | | Green | | | | | | |
| M 3-7 | 400 | Yes | 70 | Yellow | Melting | 11 | Round | Light | Pink | 9 | Semi | No | Acid | Fertile |
| | | | | | | | | Yellow | | | Cling | | | |
| M 3-7 | 400 | Yes | 70 | Yellow | Melting | 11 | Round | Light | Pink | 9 | Semi | No | Acid | Fertile |
| | | | | | | | | Yellow | | | Cling | | | |
| Sunbob | 250 | No | 70 | Yellow | Melting | 10\11 | Elon- | Light | Pink | 9 | Cling | Yes | Acid | Fertile |
| | | | | | | | gate | Yellow | | | | | | |
| Fla 82-25N | 200 A | No | 80 | Yellow | Melting | 10\11 | Round | Light | Pink | 10 | Semi Free | No | Acid | Male |
| | | | | | | | | Green | | | | | | Sterile |
| M 3-8 | 400 | Yes | 70 | Yellow | Melting | 11\12 | Round | Light | Pink | 9 | Cling | No | Acid | Fertile |
| | | | | | | | | Yellow | | | | | | |
| M 3-7 | 400 | Yes | 70 | Yellow | Melting | 11 | Round | Light | Pink | 9 | Semi | No | Acid | Fertile |
| | | | | | | | | Yellow | | | Cling | | | |
| M3-6 | 400 | Yes | 70 | Yellow | Melting | 11 | Round | Light | Pink | 9 | Cling | No | Acid | Fertile |
| | | | | | | | | Yellow | | | | | | |
| Fla 82-12 | 200 | Yes | 70 | Yellow | Melting | 10 | Pointed | Light | Pink | 7 | Cling | No | Acid | Male |
| | | | | | | | | Green | | | | | | Sterile |
| Fla 84-22 | 250 | Yes | 30 | White | Melting | 12 | Pointed | Dark | Pink | 6 | Cling | Yes | Sub - | Fertile |
| | | | | | | | | Green | | | | | Acid | |
| Fla 82-25N | 200 A | No | 80 | Yellow | Melting | 10\11 | Round | Light | Pink | 10 | Semi Free | No | Acid | Male |
| | | | | | | | | Green | | | | | | Sterile |
| Fla 4-3 | 200 | Yes | 30 | Yellow | Melting | 11 | Pointed | Light | Pink/ | 5 | Cling | Yes | Acid | Fertile |
| | | | | | | | | Green | Multi | | | | | |
| | | | | | | | | | double | | | | | |
| VV 91-1 | 150 | Yes | 30 | White | Melting | 11 | Pointed | Dark | Pink/ | 5 | Cling | Yes | Acid | Fertile |
| Marie | | | | | | | | Green | Multi | | | | | |
| | | | | | | | | | double | | | | | |

†Chill requirement - minimum hours required at less than 7° C. to break dormancy in 50% of the flower/leaf buds.

TABLE 5
Selected F₁ progeny from plants of Table 4

| | | | BLUSH | FLESH | BRIX | LEAF | STONE |
|---|---|---|---|---|---|---|---|
| SELECTIONS | CHILL* | PUBESENCE | (%) | COLOUR | (%) | COLOUR | ADHESION |
| 99-5NW | 300 | NO | 80+ | WHITE | 11-12 | DARK | CLING |
| | | | | | | GREEN | |
| 94-22N | 250 | NO | 70+ | YELLOW | 10-11 | LIGHT | CLING |
| | | | | | | GREEN | |
| 94-30NW | 250 | NO | 70+ | WHITE | 10-11 | DARK | CLING |
| | | | | | | GREEN | |
| 94-66WF | 250† | YES | 60+ | WHITE | 10-11 | DARK | FREE |
| | | | | | | GREEN | |
| 94-29NW | 250 | NO | 60+ | WHITE | 10-12 | DARK | CLING |
| | | | | | | GREEN | |
| 94-10F | 200 | YES | 60+ | YELLOW | 10-12 | LIGHT | FREE |
| | | | | | | GREEN | |
| 94-75W | 300 | YES | 60+ | WHITE | 9-11 | DARK | CLING |
| | | | | | | GREEN | |
| 94-54NF | 300 | NO | 60+ | YELLOW | 10-12 | LIGHT | CLING |
| | | | | | | GREEN | |
| 95-11F | 350 | YES | 60+ | YELLOW | 10-12 | LIGHT | FREE |
| | | | | | | GREEN | |
| 94-53 | 350 | YES | 60+ | YELLOW | 10-12 | LIGHT | CLING |
| | | | | | | GREEN | |
| 95-9 | 350 | YES | 60+ | YELLOW | 10-12 | LIGHT | CLING |
| | | | | | | GREEN | |
| 95-3 | 350 | YES | 60+ | YELLOW | 10-12 | LIGHT | CLING |
| | | | | | | GREEN | |
| 95-5 | 350 | YES | 60+ | YELLOW | 10-12 | LIGHT | CLING |
| | | | | | | GREEN | |
| 95-4F | 350 | YES | 60+ | YELLOW | 10-12 | LIGHT | FREE |
| | | | | | | GREEN | |
| 94-15 | 200 | YES | 90 | YELLOW | 10-12 | LIGHT | CLING |
| | | | | | | GREEN | |
| 94-24NW | 250 | NO | 60+ | WHITE | 10-12 | DARK | CLING |
| | | | | | | GREEN | |
| 94-72WF | 300 | YES | 60+ | WHITE | 10-12 | DARK | FREE |
| | | | | | | GREEN | |
| 94-6 | 200 | YES | 60+ | YELLOW | 10-12 | LIGHT | CLING |
| | | | | | | GREEN | |
| 94-76N | 300 | NO | 60+ | YELLOW | 10-12 | LIGHT | CLING |
| | | | | | | GREEN | |
| 94-25N | 250 | NO | 60+ | YELLOW | 10-12 | LIGHT | CLING |
| | | | | | | GREEN | |
| 94-47N | 250 | NO | 60+ | YELLOW | 10-12 | LIGHT | CLING |
| | | | | | | GREEN | |
| 94-11N | 250 | NO | 60+ | YELLOW | 10-12 | LIGHT | CLING |
| | | | | | | GREEN | |
| 95-11WF | 350 | YES | 60+ | WHITE | 10-12 | DARK | FREE |
| | | | | | | GREEN | |
| 95-7N | 350 | NO | 60+ | YELLOW | 10-12 | LIGHT | CLING |
| | | | | | | GREEN | |
| 94-30N | 250 | NO | 60+ | YELLOW | 10-12 | LIGHT | CLING |
| | | | | | | GREEN | |
| 94-27NW | 250 | NO | 60+ | WHITE | 10-12 | DARK | CLING |
| | | | | | | GREEN | |
| 94-10 | 200 | YES | 60+ | YELLOW | 10-12 | LIGHT | CLING |
| | | | | | | GREEN | |
| 94-19N | 250 | NO | 60+ | YELLOW | 10-12 | LIGHT | CLING |
| | | | | | | GREEN | |
| 94-20 | 250 | YES | 60+ | YELLOW | 10-12 | LIGHT | CLING |
| | | | | | | GREEN | |
| 94-26NW | 250+ | NO | 60+ | WHITE | 10-12 | DARK | CLING |
| | | | | | | GREEN | |
| 94-8 | 200 | YES | 60+ | YELLOW | 10-12 | LIGHT | CLING |
| | | | | | | GREEN | |
| 95-6NF | 200 | NO | 60+ | YELLOW | 10-12 | LIGHT | FREE |
| | | | | | | GREEN | |
| 94-1N | 150 | NO | 70+ | YELLOW | 10-12 | LIGHT | CLING |
| | | | | | | GREEN | |

*Chill requirement - minimum hours required at less than 7° C. to break dormancy in 50% of the flower/leaf buds.
†Long fruit development period.

### Example 5

Male sterile nectarine FLA 82-25N× polymix open pollination gave UWS P 94-5.

UWS P 94-5 was then crossed with supersweet unnamed white fleshed seedling as pollen parent using UWS P 94-5 as pollen parent. This cross generated many seedlings being evaluated and gave rise to a polymix.

| | | | |
|---|---|---|---|
| Nectarines: | UWS 98-12 NWFH | homozygous | Nectarine |
| | | | White flesh |
| | | | No red around pit |
| | | | Round shape |
| | | | High Blush |
| | UWS 98-4 NYH | homozygous | Nectarine |
| | | | Yellow Flesh |
| | | | Round Shape |
| | UWS 98-4 NW | homozygous | Nectarine |
| | | | White Flesh |
| | | | Round Shape |

These are now all homozygous breeding lines and have been proven by selfing each selection or seedling from each parent, and by knowing the dominance relationship of each characteristic.

### Example 6

A simple version of a method of the invention for generating a male sterile peach or nectarine breeding line incorporating at least low chill requirement as a target trait derived from a non-domesticated species related to peach or nectarine may be as follows:

a) select a male sterile peach or nectarine variety which may also be of known genotype for one or more desired commercially important traits such as fruit flesh or skin colour, melting or non-melting flesh, freestone or clingstone, tree habit, spur habit, suture presence/absence, or a combination thereof;

b) select a plant sufficiently related to peaches or nectarines so as to be able to hybridise with the plant of step (a), which has low or no chilling requirement, and which optionally expresses or comprises an allele for at least one other target trait, such as disease/pest resistance. The ability of this plant to hybridise with the plant selected in step (a) is most effectively determined by cladistic analysis using analysis of extracted genetic material, for example as described below;

c) cross the plant of step (a) with the plant of step (b) artificially using stored pollens (the flowering times of the plants selected in steps (a) and (b) will not necessarily coincide);

d) select progeny resulting from the cross of step (c) which have at least one allele associated with male sterility, which exhibit low chill requirement, optionally in combination with at least one allele for at least one other target trait, and which preferably are of known genotype for one or more desired inheritable traits. This selection may be made by observation of the phenotypes of the progeny themselves, progeny thereof resulting from self-fertilisation or crossing with another plant of known genotype, by genetic marker analysis, or by other suitable method.

If the F₁ progeny do not possess sufficiently desirable commercial traits, one or more sequential back-crosses with a commercial variety which is of known genotype with respect to a set of desired inheritable traits, testing the progeny at all stages for inclusion of at least one allele for male sterility and at least one allele for low chill and, optionally other target trait(s), until the resulting progeny has incorporated a desirable set of commercially important traits, along with at least one allele for male sterility and at least one allele for low chill and, optionally one or more other target traits. Selected plants from the resulting progeny may then be allowed to self fertilise, or be crossed with a mixture of pollen from the selected progeny;

e) selecting progeny plants which are homozygous for male sterility, which express low chill requirements, and optionally one or more other target traits and which are homozygous for a desired set of inheritable commercially important traits.

The present invention may be used to provide, for example, the following advantages:

1. Creation of low chill, super-sweet peach and nectarine varieties. Can use combination of peaches and nectarines in the one block but incorporation of a male sterile gene into the peach/nectarine in the growing block;

2. Using a low chill super-sweet nectarine to create a suite of cultivars that incorporate varying fruit development periods, therefore giving market availability over an extended period of time.

3. Incorporation of a designed low or high chill character to enable optimal growth/fruiting for particular climatic conditions, and/or allow for climate changes, e.g. global warming.

4. Balance of vegetative growth relative to fruit character (e.g. use of spur genes from plums into peaches).

5. Increased adaptability and grower friendliness using natural techniques.

6. Creation of improved root stocks—e.g. peach×plum (“Pleach”) which should be more adaptable for wet conditions—an issue for root stocks that get root rot due to periodic waterlogging (site specific).

It will be appreciated that, although specific embodiments of the invention have been described herein for the purpose of illustration only, various modifications may be made without deviating from the spirit and scope of the invention as defined in the following claims.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. A method for generating a woody perennial breeding line, comprising:
a) selecting one or more woody perennial plants comprising at least one allele associated with male sterility;
b) selecting one or more woody perennial plants which are capable of hybridisation with the plant(s) selected in step (a) and which comprise at least one allele associated with at least one target inheritable trait;
c) crossing the one or more plants selected in step (a) with the one or more plants selected in step (b);
d) selecting progeny plants which have one or more desired traits and which comprise at least one allele associated with male sterility and at least one allele associated with the at least one target trait;
e) selecting progeny plants which are homozygous for male sterility, which comprise at least one allele associated with the at least one target trait and which have one or more desired traits.

85. The method of claim 84, wherein step (d) further comprises allowing at least one of the progeny plants selected at step (d) which is heterozygous for male sterility to self-fertilise to create a subsequent progeny for selection at step (e).

86. The method of claim 84, wherein the flowers of one or more of the progeny selected at step (d) are fertilised with a mixture of pollen obtained from a plurality of the progeny selected at step (d) to create a subsequent progeny for selection at step (e).

87. The method of claim 84, wherein step (d) further comprises crossing at least one of the progeny plants selected at step (d) with one or more woody perennial plants of known genotype with respect to a desired set of inheritable traits and which comprises at least one allele associated with male sterility to create a subsequent progeny for selection at step (e).

88. The method of claim 84, wherein the one or more woody perennial plants selected in step (a) are male sterile.

89. The method of claim 86, wherein the one or more woody perennial plants of step (a) are grown in an isolated block, with the one or more woody perennial plants of step (b).

90. The method of claim 84, wherein (i) a plurality of woody perennial plants are selected in step (a), and the flowers of the one or more woody perennial plants selected in step (b) are fertilised with a mixture of pollen collected from the woody perennial plants of step (a) or (ii) a plurality of woody perennial plants are selected in step (b), and the flowers of the one or more woody perennial plants selected in step (a) are fertilised with a mixture of pollen collected from the woody perennial plants of step (b).

91. The method of claim 89, wherein the plurality of woody perennial plants selected in step (a), step (b) or both step (a) and step (b) comprise more than one variety of woody perennial plant.

92. The method of claim 90, wherein the plurality of woody perennial plants selected in step (a), step (b) or both step (a) and step (b) comprise more than one variety of woody perennial plant.

93. The method of claim 84, wherein one woody perennial plant is selected in step (a), step (b) or both step (a) and step (b).

94. The method of claim 84, wherein the one or more woody perennial plants selected in step (a), step (b) or both step (a) and step (b) are of known genotype for a set of desired inheritable traits.

95. The method of claim 84, wherein the one or more woody perennial plants selected in step (a), step (b) or both step (a) and step (b) are homozygous for each trait within a set of desired inheritable traits.

96. The method of claim 84, wherein one or more of the plants of step (b) are heterozygous for male sterility.

97. The method of claim 84, wherein the one or more plants of step (b) are derived from a plant comprising fewer chromosomes than the plant of step (a) by increasing its ploidy.

98. The method of claim 97, wherein a plurality of plants are selected in step (a), step (b), or both, and a mixture of pollens from these plants are employed in the cross of step (c).

99. The method of claim 84, wherein step (d) further comprises one or more sequential back-crosses of one or more progeny plants selected at step (d) with one or more woody perennial plants of known genotype with respect to a desired set of inheritable traits, and selecting resulting progeny plants which comprise at least one allele coding for male sterility and at least one allele associated with the at least one target trait and which are of known genotype for the desired set of inheritable traits.

100. The method of claim 99, wherein the one or more woody perennial plants used for backcrossing to the progeny of step (d) are homozygous for each trait within a set of desired inheritable traits.

101. The method of claim 99, wherein the one or more woody perennial plants used for backcrossing to the progeny of step (d) are heterozygous for male sterility.

102. The method of claim 99, wherein the one or more woody perennial plants used for backcrossing to the progeny of step (d) are the same, or are of the same variety as the woody perennial plant of step (a).

103. The method of claim 99, wherein the one or more woody perennial plants used for backcrossing to the progeny of step (d) are the same, or are of the same variety as the one or more woody perennial plants of step (b).

104. The method of claim 84, wherein at least one target trait comprises low chill requirement, disease/pest resistance, or both.

105. The method of claim 84, wherein desired inheritable traits are selected from one or more of the group comprising: low chill requirement; high chill requirement; disease/pest resistance; fruit development period; fruit acidity; fruit shape; fruit size; fruit flesh texture; fruit total solids (sugars); fruit pigmentation; fruit flesh pigmentation; fruit skin pubescence; stone adhesion to the fruit; tree habit; tree size; tree growth rate; spur morphology/habit; pedicel length; pedicel thickness; suture presence/absence.

106. The method of claim 84, wherein all the plants are of the genus Prunus.

107. The method of claim 106, wherein the woody perennial plant of step (a) is selected from Prunus persica, P. persica var nucipersica, P. persica var nectarine, P. salicina, P. avium, P. cerasus, P. domestics, P. amygdalus, and P. armeniaca.

108. The method of claim 106, wherein the one or more woody perennial plants of step (a), step (b) or both step (a) and step (b) is/are selected from peach or nectarine varieties.

109. The method of claim 106, wherein the one or more woody perennial plants of step (a), step (b) or both step (a) and step (b) is/are selected from plum or apricot varieties.

110. The method of claim 109, wherein step (d) further comprises one or more sequential back-crosses of one or more selected progeny plants with the pollen of a plurality of plum or apricot varieties.

111. The method of claim 106, wherein the one or more woody perennial plants of step (a) are of a different species to the one or more woody perennial plants of step (b), but the plants of step (a) and step (b) are sufficiently related to be able to hybridise.

112. The method of claim 106, wherein step (d) further comprises one or more sequential back-crosses of one or more selected progeny plants with one or more woody perennial plants which are selected from peach, nectarine, plum cherry, almond or apricot varieties.

113. The method of claim 84, wherein the resulting woody perennial breeding line is male sterile, homozygous for said one or more desired inheritable traits, and homozygous for at least one allele associated with said at least one target trait.

114. A male sterile woody perennial plant breeding line comprising one or more target inheritable traits and one or more desired inheritable traits, generated by directed breeding to transfer male sterility from a woody perennial plant to a woody perennial plant having one or more desired inheritable traits.

115. The male sterile woody perennial plant breeding line of claim 114, which is a Prunus variety selected from peach, nectarine, plum, cherry, apricot or almond varieties.

116. The male sterile woody perennial plant breeding line of claim 114, which is an interspecific hybrid.

117. The male sterile woody perennial plant breeding line of claim 114, wherein the at least one target inheritable trait and the one or more desired inheritable traits are selected from one or more of the group comprising: low chill requirement; high chill requirement; disease/pest resistance; fruit development period; fruit acidity; fruit shape; fruit size; fruit flesh texture; fruit total solids (sugars); fruit pigmentation; fruit flesh pigmentation; fruit skin pubescence; stone adhesion to the fruit; tree habit; tree size; tree growth rate; spur morphology/habit; pedicel length; pedicel thickness; suture presence/absence.

118. The male sterile woody perennial plant breeding line of claim 117, wherein the one or more target traits comprise low chill requirement, disease/pest resistance, or both.

119. The male sterile woody perennial plant breeding line of claim 114, which is male sterile, homozygous for a desired set of inheritable traits, and homozygous for at least one allele associated with a target trait.

120. A method for generating a woody perennial variety comprising one or more target inheritable traits and a desired set of inheritable traits, comprising crossing a first selected woody perennial plant variety with a desired set of inheritable traits, or a group of plants sharing a set of desired inheritable traits, with a second selected woody perennial plant which is homozygous for male sterility, and homozygous for one or more target traits.

121. The method of claim 120, wherein the second plant is of known genotype with respect to the desired set of inheritable traits of the first selected woody perennial plant or group of plants sharing a set of desired inheritable traits.

122. The method of claim 120, wherein the first selected woody perennial plant or group of plants, or the second plant, or both the first plant or group of plants and the second plant is/are homozygous for each of the traits in the desired set of inheritable traits.

123. The method of claim 120, wherein the second woody perennial plant is a male sterile woody perennial plant breeding line according to claim 119.

124. The method of claim 120, wherein the first selected woody perennial plant variety with a desired set of inheritable traits, or group of plants sharing a set of desired inheritable traits, are planted in an orchard, surrounding the second plant.

125. The method of claim 120, wherein the flowers of the second plant are artificially fertilised with pollen from the first selected woody perennial plant variety with a desired set of inheritable traits, or a mixture of pollen from the group of plants sharing a set of desired inheritable traits.

126. The method of claim 120, wherein all of the plants are selected from Prunus plants which are, or are essentially peach, nectarine, plum, cherry, apricot or almond varieties.

127. A woody perennial plant variety expressing at least one target inheritable trait and a set of desired inheritable traits generated by crossing a male sterile woody perennial plant breeding line according to claim 120 having said at least one target inheritable trait with a woody perennial plant variety having said set of desired inheritable traits but lacking said at least one target inheritable trait.

128. The woody perennial plant variety of claim 127, which is selected from Prunus plants which are, or are essentially peach, nectarine, plum, cherry, apricot or almond varieties.

129. The woody perennial plant variety of claim 127, which expresses at least low chill, disease/pest resistance, or both as target traits.
